SEQUENCE LISTING (78 sequences)

SEQ ID NO: 1 - RNA, 22 nt, Homo sapiens
  source 1..22: mol_type = genomic RNA
tggaatgtaaagaagtatgtat

SEQ ID NO: 2 - RNA, 23 nt, Homo sapiens
  source 1..23: mol_type = genomic RNA
tggagacgcggccctgttggagt

SEQ ID NO: 3 - RNA, 22 nt, Homo sapiens
  source 1..22: mol_type = genomic RNA
taaggcacgcggtgaatgccaa

SEQ ID NO: 4 - RNA, 23 nt, Homo sapiens
  source 1..23: mol_type = genomic RNA
tgtagtgtttcctactttatgga

SEQ ID NO: 5 - RNA, 23 nt, Homo sapiens
  source 1..23: mol_type = genomic RNA
tgagaactgaattccataggctg

SEQ ID NO: 6 - RNA, 22 nt, Homo sapiens
  source 1..22: mol_type = genomic RNA
tagcagcacatcatggtttaca

SEQ ID NO: 7 - RNA, 21 nt, Homo sapiens
  source 1..21: mol_type = genomic RNA
ttcaagtaattcaggataggt

SEQ ID NO: 8 - RNA, 22 nt, Homo sapiens
  source 1..22: mol_type = genomic RNA
cattgcacttgtctcggtctga

SEQ ID NO: 9 - RNA, 23 nt, Homo sapiens
  source 1..23: mol_type = genomic RNA
taggcagtgtcattagctgattg

SEQ ID NO: 10 - RNA, 22 nt, Homo sapiens
  source 1..22: mol_type = genomic RNA
tattgcacttgtcccggcctgt

SEQ ID NO: 11 - RNA, 22 nt, Homo sapiens
  source 1..22: mol_type = genomic RNA
tcgtaccgtgagtaataatgcg

SEQ ID NO: 12 - RNA, 22 nt, Homo sapiens
  source 1..22: mol_type = genomic RNA
tcagtgcactacagaactttgt

SEQ ID NO: 13 - RNA, 22 nt, Homo sapiens
  source 1..22: mol_type = genomic RNA
tagcttatcagactgatgttga

SEQ ID NO: 14 - RNA, 22 nt, Homo sapiens
  source 1..22: mol_type = genomic RNA
agcccctgcccaccgcacactg

SEQ ID NO: 15 - RNA, 23 nt, Homo sapiens
  source 1..23: mol_type = genomic RNA
agctacattgtctgctgggtttc

SEQ ID NO: 16 - RNA, 22 nt, Homo sapiens
  source 1..22: mol_type = genomic RNA
ttcaagtaatccaggataggct

SEQ ID NO: 17 - RNA, 22 nt, Homo sapiens
  source 1..22: mol_type = genomic RNA
tagcaccatctgaaatcggtta

SEQ ID NO: 18 - RNA, 23 nt, Homo sapiens
  source 1..23: mol_type = genomic RNA
tagcaccatttgaaatcagtgtt

SEQ ID NO: 19 - RNA, 22 nt, Homo sapiens
  source 1..22: mol_type = genomic RNA
tagcaccatttgaaatcggtta

SEQ ID NO: 20 - RNA, 22 nt, Homo sapiens
  source 1..22: mol_type = genomic RNA
tgtaaacatcctcgactggaag

SEQ ID NO: 21 - RNA, 22 nt, Homo sapiens
  source 1..22: mol_type = genomic RNA
tggcagtgtcttagctggttgt

SEQ ID NO: 22 - RNA, 23 nt, Homo sapiens
  source 1..23: mol_type = genomic RNA
gtccagttttcccaggaatccct

SEQ ID NO: 23 - RNA, 22 nt, Homo sapiens
  source 1..22: mol_type = genomic RNA
ctggtacaggcctgggggacag

SEQ ID NO: 24 - RNA, 22 nt, Homo sapiens
  source 1..22: mol_type = genomic RNA
ctcctacatattagcattaaca

SEQ ID NO: 25 - RNA, 22 nt, Homo sapiens
  source 1..22: mol_type = genomic RNA
tagcagcacgtaaatattggcg

SEQ ID NO: 26 - RNA, 23 nt, Homo sapiens
  source 1..23: mol_type = genomic RNA
taaggtgcatctagtgcagatag

SEQ ID NO: 27 - RNA, 23 nt, Homo sapiens
  source 1..23: mol_type = genomic RNA
aacattcattgctgtcggtgggt

SEQ ID NO: 28 - RNA, 22 nt, Homo sapiens
  source 1..22: mol_type = genomic RNA
tgtcagtttgtcaaatacccca

SEQ ID NO: 29 - RNA, 21 nt, Homo sapiens
  source 1..21: mol_type = genomic RNA
cctgttctccattacttggct

SEQ ID NO: 30 - RNA, 21 nt, Homo sapiens
  source 1..21: mol_type = genomic RNA
tcactcctctcctcccgtctt

SEQ ID NO: 31 - RNA, 22 nt, Homo sapiens
  source 1..22: mol_type = genomic RNA
gggagccaggaagtattgatgt

SEQ ID NO: 32 - RNA, 23 nt, Homo sapiens
  source 1..23: mol_type = genomic RNA
tgtgcttgctcgtcccgcccgca

SEQ ID NO: 33 - RNA, 21 nt, Homo sapiens
  source 1..21: mol_type = genomic RNA
tctgagagagctcgatggcag

SEQ ID NO: 34 - RNA, 23 nt, Homo sapiens
  source 1..23: mol_type = genomic RNA
tgagcgcctcgacgacagagccg

SEQ ID NO: 35 - RNA, 21 nt, Homo sapiens
  source 1..21: mol_type = genomic RNA
tgtcttgcaggccgtcatgca

SEQ ID NO: 36 - RNA, 23 nt, Homo sapiens
  source 1..23: mol_type = genomic RNA
agcagcattgtacagggctatga

SEQ ID NO: 37 - DNA, 62 bp, Homo sapiens
  source 1..62: mol_type = genomic DNA
cacagatgatgaacttattgacgggcggacagaaactgtgtgctgattgtcacgttctgatt

SEQ ID NO: 38 - DNA, 763 bp, Homo sapiens
  source 1..763: mol_type = genomic DNA
gctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccccttcattgacctcaactacatggtttacatgttccaatatgattccacccatggcaaccatcaatgaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacca

SEQ ID NO: 39 - DNA, 25 bp, synthetic construct
  misc_feature 23: note = RNA
  source 1..25: mol_type = other DNA
  misc_feature 1..22: note = DNA
  misc_feature 24..25: note = DNA
gccactggaatgtaaagaagtatgt

SEQ ID NO: 40 - DNA, 20 bp, synthetic construct
  misc_feature 18: note = RNA
  source 1..20: mol_type = other DNA
  misc_feature 1..17: note = DNA
  misc_feature 19..20: note = DNA
ttattagacgcggccctgtt

SEQ ID NO: 41 - DNA, 22 bp, synthetic construct
  misc_feature 19: note = RNA
  source 1..22: mol_type = other DNA
  misc_feature 1..18: note = DNA
  misc_feature 20..22: note = DNA
aattaaggcacgcggtgaatgc

SEQ ID NO: 42 - DNA, 25 bp, synthetic construct
  misc_feature 23: note = RNA
  source 1..25: mol_type = other DNA
  misc_feature 1..22: note = DNA
  misc_feature 24..25: note = DNA
gccctgtagtgtttcctactttatg

SEQ ID NO: 43 - DNA, 25 bp, synthetic construct
  misc_feature 23: note = RNA
  source 1..25: mol_type = other DNA
  misc_feature 1..22: note = DNA
  misc_feature 24..25: note = DNA
gccactgagaactgaattccatagg

SEQ ID NO: 44 - DNA, 24 bp, synthetic construct
  misc_feature 22: note = RNA
  source 1..24: mol_type = other DNA
  misc_feature 1..21: note = DNA
  misc_feature 23..24: note = DNA
gcgtatagcagcacatcatggttt

SEQ ID NO: 45 - DNA, 25 bp, synthetic construct
  misc_feature 23: note = RNA
  source 1..25: mol_type = other DNA
  misc_feature 1..22: note = DNA
  misc_feature 24..25: note = DNA
cgcttcaagtaattcaggataggta

SEQ ID NO: 46 - DNA, 21 bp, synthetic construct
  misc_feature 19: note = RNA
  source 1..21: mol_type = other DNA
  misc_feature 1..18: note = DNA
  misc_feature 20..21: note = DNA
acattgcacttgtctcggtct

SEQ ID NO: 47 - DNA, 23 bp, synthetic construct
  misc_feature 21: note = RNA
  source 1..23: mol_type = other DNA
  misc_feature 1..20: note = DNA
  misc_feature 22..23: note = DNA
ctaggcagtgtcattagctgatt

SEQ ID NO: 48 - DNA, 21 bp, synthetic construct
  misc_feature 19: note = RNA
  source 1..21: mol_type = other DNA
  misc_feature 1..18: note = DNA
  misc_feature 20..21: note = DNA
gcctatattgcacttgtcccg

SEQ ID NO: 49 - DNA, 24 bp, synthetic construct
  misc_feature 22: note = RNA
  source 1..24: mol_type = other DNA
  misc_feature 1..21: note = DNA
  misc_feature 23..24: note = DNA
ggtcgtaccgtgagtaataatgcg

SEQ ID NO: 50 - DNA, 23 bp, synthetic construct
  misc_feature 21: note = RNA
  source 1..23: mol_type = other DNA
  misc_feature 1..20: note = DNA
  misc_feature 22..23: note = DNA
acctcagtgcactacagaacttt

SEQ ID NO: 51 - DNA, 25 bp, synthetic construct
  misc_feature 23: note = RNA
  source 1..25: mol_type = other DNA
  misc_feature 1..22: note = DNA
  misc_feature 24..25: note = DNA
gccactagcttatcagactgatgtt

SEQ ID NO: 52 - DNA, 16 bp, synthetic construct
  misc_feature 14: note = RNA
  source 1..16: mol_type = other DNA
  misc_feature 1..13: note = DNA
  misc_feature 15..16: note = DNA
atgcccaccgcacact

SEQ ID NO: 53 - DNA, 22 bp, synthetic construct
  misc_feature 20: note = RNA
  source 1..22: mol_type = other DNA
  misc_feature 1..19: note = DNA
  misc_feature 21..22: note = DNA
tagctacattgtctgctgggtt

SEQ ID NO: 54 - DNA, 26 bp, synthetic construct
  misc_feature 24: note = RNA
  source 1..26: mol_type = other DNA
  misc_feature 1..23: note = DNA
  misc_feature 25..26: note = DNA
ccacttcaagtaatccaggataggct

SEQ ID NO: 55 - DNA, 23 bp, synthetic construct
  misc_feature 21: note = RNA
  source 1..23: mol_type = other DNA
  misc_feature 1..20: note = DNA
  misc_feature 22..23: note = DNA
gagtagcaccatctgaaatcggt

SEQ ID NO: 56 - DNA, 23 bp, synthetic construct
  misc_feature 21: note = RNA
  source 1..23: mol_type = other DNA
  misc_feature 1..20: note = DNA
  misc_feature 22..23: note = DNA
ccctagcaccatttgaaatcagt

SEQ ID NO: 57 - DNA, 23 bp, synthetic construct
  misc_feature 21: note = RNA
  source 1..23: mol_type = other DNA
  misc_feature 1..20: note = DNA
  misc_feature 22..23: note = DNA
gagtagcaccatttgaaatcggt

SEQ ID NO: 58 - DNA, 24 bp, synthetic construct
  misc_feature 22: note = RNA
  source 1..24: mol_type = other DNA
  misc_feature 1..21: note = DNA
  misc_feature 23..24: note = DNA
gactgtaaacatcctcgactggaa

SEQ ID NO: 59 - DNA, 21 bp, synthetic construct
  misc_feature 19: note = RNA
  source 1..21: mol_type = other DNA
  misc_feature 1..18: note = DNA
  misc_feature 20..21: note = DNA
ttggcagtgtcttagctggtt

SEQ ID NO: 60 - DNA, 23 bp, synthetic construct
  misc_feature 21: note = RNA
  source 1..23: mol_type = other DNA
  misc_feature 1..20: note = DNA
  misc_feature 22..23: note = DNA
ttgtccagttttcccaggaatcc

SEQ ID NO: 61 - DNA, 20 bp, synthetic construct
  misc_feature 18: note = RNA
  source 1..20: mol_type = other DNA
  misc_feature 1..17: note = DNA
  misc_feature 19..20: note = DNA
tatctggtacaagcctgggg

SEQ ID NO: 62 - DNA, 26 bp, synthetic construct
  misc_feature 23: note = RNA
  source 1..26: mol_type = other DNA
  misc_feature 1..22: note = DNA
  misc_feature 24..26: note = DNA
ccacctcctacatattagcattaaca

SEQ ID NO: 63 - DNA, 24 bp, synthetic construct
  misc_feature 22: note = RNA
  source 1..24: mol_type = other DNA
  misc_feature 1..21: note = DNA
  misc_feature 23..24: note = DNA
ggtagcagcacgtagatattggcg

SEQ ID NO: 64 - DNA, 21 bp, synthetic construct
  misc_feature 19: note = RNA
  source 1..21: mol_type = other DNA
  misc_feature 1..18: note = DNA
  misc_feature 20..21: note = DNA
gggtgtaaggtgcatctagtg

SEQ ID NO: 65 - DNA, 18 bp, synthetic construct
  misc_feature 16: note = RNA
  source 1..18: mol_type = other DNA
  misc_feature 1..15: note = DNA
  misc_feature 17..18: note = DNA
tcattgctgtcggtgggt

SEQ ID NO: 66 - DNA, 23 bp, synthetic construct
  misc_feature 21: note = RNA
  source 1..23: mol_type = other DNA
  misc_feature 1..20: note = DNA
  misc_feature 22..23: note = DNA
ttgtcagtttgtcaaatacccca

SEQ ID NO: 67 - DNA, 23 bp, synthetic construct
  misc_feature 21: note = RNA
  source 1..23: mol_type = other DNA
  misc_feature 1..20: note = DNA
  misc_feature 22..23: note = DNA
ccacccctgttctccattacttg

SEQ ID NO: 68 - DNA, 19 bp, synthetic construct
  misc_feature 17: note = RNA
  source 1..19: mol_type = other DNA
  misc_feature 1..16: note = DNA
  misc_feature 18..19: note = DNA
tcactcctctcctcccgtc

SEQ ID NO: 69 - DNA, 21 bp, synthetic construct
  misc_feature 19: note = RNA
  source 1..21: mol_type = other DNA
  misc_feature 1..18: note = DNA
  misc_feature 20..21: note = DNA
gggagccaggaagtattgatg

SEQ ID NO: 70 - DNA, 18 bp, synthetic construct
  misc_feature 16: note = RNA
  source 1..18: mol_type = other DNA
  misc_feature 1..15: note = DNA
  misc_RNA 17..18: note = DNA
atgtgcttgctcgtcccg

SEQ ID NO: 71 - DNA, 21 bp, synthetic construct
  misc_feature 19: note = RNA
  source 1..21: mol_type = other DNA
  misc_feature 1..18: note = DNA
  misc_feature 20..21: note = DNA
tctcctacgctgctctctcac

SEQ ID NO: 72 - DNA, 20 bp, synthetic construct
  misc_feature 18: note = RNA
  source 1..20: mol_type = other DNA
  misc_feature 1..17: note = DNA
  misc_feature 19..20: note = DNA
tatagcgcctcgacgacaga

SEQ ID NO: 73 - DNA, 21 bp, synthetic construct
  misc_feature 19: note = RNA
  source 1..21: mol_type = other DNA
  misc_feature 1..18: note = DNA
  misc_feature 20..21: note = DNA
ttatgtcttgcaggccgtcat

SEQ ID NO: 74 - DNA, 18 bp, synthetic construct
  misc_feature 16: note = RNA
  source 1..18: mol_type = other DNA
  misc_feature 1..15: note = DNA
  misc_RNA 17..18: note = DNA
agcagcattgtacagggc

SEQ ID NO: 75 - DNA, 21 bp, synthetic construct
  misc_feature 19: note = RNA
  source 1..21: mol_type = other DNA
  misc_feature 1..18: note = DNA
  misc_RNA 20..21: note = DNA
aactgtgtgctgattgtcacg

SEQ ID NO: 76 - DNA, 17 bp, synthetic construct
  misc_feature 15: note = RNA
  source 1..17: mol_type = other DNA
  misc_feature 1..14: note = DNA
  misc_RNA 16..17: note = DNA
gagccgcaccttgtcat

SEQ ID NO: 77 - DNA, 39 bp, synthetic construct
  source 1..39: mol_type = other DNA
gttgattttttctttttcaactttttttttttttttttt

SEQ ID NO: 78 - DNA, 39 bp, synthetic construct; identical to SEQ ID NO: 77
  source 1..39: mol_type = other DNA

(19) United States
(12) Patent Application Publication
(10) Pub. No.: US 2024/0209450 A1
(43) Pub. Date: Jun. 27, 2024

(54) Multiple biomarkers for early diagnosis of multi cancer and uses thereof

(71) Applicant: NURIBIO CO., LTD, Sejong-si (KR)

(72) Inventor: Young Hyean NAM, Suwon-si (KR)

(21) Appl. No.: 18/495,951

(22) Filed: Oct. 27, 2023

Related U.S. Application Data
(60) Provisional application No. 63/420,159, filed on Oct. 28, 2022.

(30) Foreign Application Priority Data
Oct. 31, 2022 (KR) 10-2022-0142093

(51) Int. Cl. C12Q 1/6886; G16B 25/10; G16H 50/20

(52) U.S. Cl. CPC C12Q 1/6886; G16B 25/10; G16H 50/20; C12Q 2600/112; C12Q 2600/118; C12Q 2600/178

(57) ABSTRACT

The present invention relates to multiple biomarkers for diagnosis of multiple types of cancer and the use thereof. Specifically, the present invention relates to multiple biomarkers for early diagnosis or prognostic prediction of multiple types of cancer, including lung cancer, pancreatic cancer, and colorectal cancer, a composition or a kit for diagnosis or prognostic prediction of multiple types of cancer comprising the multiple biomarkers, and a method of providing information necessary for early diagnosis or prognostic prediction of multiple cancers using the multiple biomarkers or the composition. According to the present invention, it is possible to provide information on the diagnosis and prognosis of multiple types of cancer with high specificity, sensitivity and accuracy by a single non-invasive analysis method.

### REFERENCE TO AN ELECTRONIC SEQUENCE LISTING

The contents of the electronic sequence listing (JPK20230115US_SEQ.xml; Size: 113,171 bytes; and Date of Creation: Mar. 5, 2024) is herein incorporated by reference in its entirety. The contents of the electronic sequence listing in no way introduces new matter into the specification.

### BACKGROUND

### 1. Technical Field

The present invention relates to multiple biomarkers for early diagnosis or prognostic prediction of multi cancer, including lung cancer, pancreatic cancer, and colorectal cancer, and the use thereof. More specifically, the present invention relates to multiple biomarkers for early diagnosis or prognostic prediction of multiple types of cancer, including lung cancer, pancreatic cancer, and colorectal cancer, a composition or a kit for diagnosis or prognostic prediction of multiple types of cancer comprising the multiple biomarkers, and a method of providing information necessary for early diagnosis or prognostic prediction of multiple types of cancer using the multiple biomarkers or the composition.

### 2. Related Art

Cancers are diseases caused by abnormal cell growth and are classified into benign tumor and malignant tumor. Benign tumors grow relatively slowly and do not metastasize, whereas malignant tumors grow faster than benign tumors and can metastasize to other organs in the body. A disease commonly called cancer refers to a malignant tumor.

Lung cancer ranks first in cancer mortality in Korea and accounts for one in five cancer deaths in Korea (22.8%). According to recent data from the Korea National Statistical Office, it has been found that the mortalities of gastric cancer, liver cancer and the like tend to decrease compared to 10 years ago, while the mortality of lung cancer tends to increase by about 20%. Despite the remarkable development of modern medicine, the incidence and mortality rates of lung cancer have continuously increased. In cases of lung cancer, there are little subjective symptoms at an early stage, and thus lung cancer is detected at a late stage in many cases. Therefore, there is a problem in that it is difficult to apply various therapies after the diagnosis of the disease. Therefore, if lung cancer is detected through medical examination at an early state when treatment thereof is possible, before symptoms thereof occur, it is possible to increase the survival rate and reduce the mortality rate.

Methods that are currently mainly used as screening tests to diagnose lung cancer include X-ray examination, CT, MRI, and the like. However, all of these methods have disadvantages in that early diagnosis is difficult, the examination method is cumbersome, and exposure to radiation may occur during regular examinations. In addition, since the above tests are usually performed after the cancer has already progressed significantly, the prognosis after treatment of the cancer is poor. Therefore, to overcome these disadvantages, there is an urgent need to develop a new method for early diagnosis of lung cancer that is simple and has a high diagnosis rate.

According to the 2015 national cancer registration statistics, pancreatic cancer is an important disease that ranks eighth in cancer incidence in Korea, and the incidence rate thereof is currently continuously increasing. Since effective anticancer drugs for pancreatic cancer have not yet been developed, surgery is currently the only curative treatment method for pancreatic cancer. However, pancreatic cancer has no characteristic symptoms and there is no method for early diagnosis thereof, and thus only about 20% of pancreatic cancer patients can actually undergo surgery, and more than 80% of pancreatic cancer patients are diagnosed with terminal cancer that is incurable. In addition, for stage 1 and 2 pancreatic cancer, surgical resection is the best therapeutic method, but the survival rate of pancreatic cancer patients who have undergone surgical resection is approximately 10 to 20%. Pancreatic cancer is a cancer with the poorest prognosis, and early detection thereof is difficult because the pancreas is a retroperitoneal organ. Even if pancreatic cancer is small (2 cm or less), pancreatic cancer cells are very highly motile, and thus they invade surrounding blood vessels, digestive tract, nerves, etc., and metastasize to nearby lymph nodes or liver. Therefore, regular diagnosis, early diagnosis, and prediction of severity of pancreatic cancer are very important.

Pancreatic cancer is diagnosed by tests using tumor markers (CA19-9), imaging tests (abdominal ultrasound test, abdominal CT, magnetic resonance imaging MRI, endoscopic ultrasound test, endoscopic retrograde cholangiopancreatography, PET, diagnostic laparoscopy), biopsy, etc. However, these diagnostic methods have low accuracy or are very inconvenient, as they cause pain to the patient. Therefore, there is a need for the development of a testing method that can easily and quickly diagnose pancreatic cancer, and new diagnostic markers for blood-based tests that are non-invasive for patients and rapid and have improved sensitivity/specificity are needed. Accordingly, biomarkers associated with poor prognosis of pancreatic cancer have been actively studied.

Pathologically, colorectal cancer is mostly adenocarcinoma, and is mainly divided into colon cancer and rectal cancer according to the site of occurrence. The frequency of occurrence by site is the highest (approximately 50%) in the lower colon, that is, the rectum. Recent studies reported that the incidence and subsequent mortality of colorectal cancer are significantly increasing due to changes in dietary habits.

Treatment of colorectal cancer is based on surgical resection, followed by chemotherapy and radiotherapy. Despite advances in surgical therapy, chemotherapy, and radiotherapy, there is no dramatic improvement in prognosis of colorectal cancer once the disease has developed. The 5-year average survival rate of colorectal cancer patients is 90% or more for stage 1, 70% or more for stage 2, 50% or more for stage 3, and 5% or less for stage 4.

As such, it can be seen that, as colorectal cancer is detected and treated at an earlier stage, the survival rate of the patient significantly increases. Therefore, a method for early diagnosis of colorectal cancer is urgently needed. Diagnosis of colorectal cancer is performed by rectal digital examination, stool occult blood examination, and colonography in patients showing symptoms related to the large intestine, and, if necessary, sigmoidoscopy and biopsy through colonoscopy are performed. However, the conventional methods described above have problems in that they have low diagnostic accuracy, cannot diagnose patients before colorectal cancer develops, and cause inconvenience to the subjects.

Biomarkers are substances that are measured for use as objective indicators of normal biological processes, pathogenesis, or pharmacological responses to treatment, and are closely related to the cause or progression of a specific disease. A method of diagnosing a disease using a biomarker has the advantage of being able to detect the disease at an early stage simply by analyzing a patient's sample, such as blood. Therefore, it is inevitable to develop biomarkers for the diagnosis of various cancers such as lung cancer, pancreatic cancer, and colorectal cancer, which are difficult to detect at an early stage by existing diagnostic methods, and the developed biomarkers are expected to play an important role in cancer diagnosis and targeted treatment.

Several single biomarkers for diagnosis of cancer such as lung cancer, pancreatic cancer, or colorectal cancer are currently being used clinically, but they have the problem of having poor diagnosis rates due to their low specificity and sensitivity. Since the values of biomarkers continuously change depending on the individual's genetic factors and living environment, and are especially closely related to the occurrence of cancer and various elements of the cancer microenvironment, it is virtually impossible to make an accurate diagnosis using only single biomarkers. Therefore, in order to diagnose lung cancer with high accuracy and sensitivity, it is required to develop new multiple biomarkers that can increase specificity and sensitivity.

Micro-RNA (miRNA) is a type of non-coding RNA having a size of 19 to 23 nt, and is known to be synthesized by a series of enzymes in the nucleus and cytoplasm [Non-Patent Document 3]. In humans, approximately 1,400 miRNAs are known, which are expected to regulate more than 60% of mRNA functions [Non-Patent Document 4]. In particular, they are known to interact with the 5′ or 3′ untranslated region (UTR) of mRNA, causing degradation or translational repression, and some miRNAs have been reported to be closely related to certain diseases, including cancer [Non-Patent Document 5].

Accordingly, the present inventors made extensive efforts to develop multiple biomarkers for simultaneous early diagnosis of multiple types of cancer, including lung cancer, pancreatic cancer, and colorectal cancer, thereby completing the present invention.

### PRIOR ART DOCUMENTS

### Non-Patent Documents

[1] Simone Donati et al (2019) , Human Circulating miRNAs Real-time qRT-PCR-based Analysis: An Overview of Endogenous Reference Genes Used for Data Normalization (Int. J. Mol. Sci. 2019, 20, 4353; doi: 10.3390/ijms 20184353

[2] Soheil Madadi et al (2019) MicroRNA expression studies: challenge of selecting reliable reference controls for data normalization (Cellular and Molecular Life Sciences volume 76, pages 3497-3514 (2019)

[3] M. Sand, The pathway of miRNA maturation, Methods in molecular biology, 1095 (2014) 3-10

[4] F. Akbari Moqadam, R. Pieters, M. L. den Boer, The hunting of targets: challenge in miRNA research, Leukemia, 27 (2013) 16-23

[5] J.K. Palanichamy, D.S. Rao, miRNA dysregulation in cancer: towards a mechanistic understanding, Frontiers in genetics, 5 (2014) 54

### SUMMARY

An object of the present invention is to provide multiple biomarkers for early diagnosis or prognostic prediction of multiple types of cancer, including lung cancer, pancreatic cancer, and colorectal cancer.

Another object of the present invention is to provide a composition or a kit for early diagnosis or prognostic prediction of multiple types of cancer comprising the multiple biomarkers.

Still another object of the present invention is to provide a method of providing information necessary for early diagnosis or prognostic prediction of multiple types of cancer using the multiple biomarkers or a composition comprising the same.

In one aspect, the present invention provides multiple biomarkers for early diagnosis or prognostic prediction of multiple types of cancer.

As used herein, the term “diagnosis” means identifying the presence or features of a pathological condition, and is included to include evaluation of the effect of treatment against a specific disease or disorder (e.g., lung cancer, pancreatic cancer, colorectal cancer), determination of a subject's susceptibility to a specific disease or disorder, determination as to whether a subject is presently affected by a specific disease or disorder, or therametrics (for example, monitoring a subject's condition to provide information as to the efficacy of therapy).

As used herein, the term “prognosis” refers to the course of a disease and whether the disease has been cured, such as the likelihood of cancer-attributable death and/or progression, including recurrence, metastatic spread, or drug resistance of cancer, such as lung cancer, pancreatic cancer, or colorectal cancer.

As used herein, “early diagnosis or prognostic prediction of multiple types of cancer” may mean identifying the development of multiple types of cancer (preferably lung cancer, pancreatic cancer, and colorectal cancer) in a subject (individual), the likelihood (risk) of developing multiple types of cancer, the likelihood of growth of multiple types of cancer (preferably lung cancer, pancreatic cancer, and colorectal cancer) tissues (or tissue growth ability, an increase in cancer cell number, or an increase in cancer tissue size), and/or the likelihood (or potential) of metastasis of multiple types of cancer (preferably lung cancer, pancreatic cancer, and colorectal cancer).

As used herein, the term “multiple types of cancer” refers to one or two or more types of cancer, and the term “cancer” preferably refers to lung cancer, colorectal cancer, and pancreatic cancer.

As used herein, the term “multiple biomarkers” refers to two or more biomarkers, and the term “biomarkers” refers to miRNAs present in blood, especially plasma.

As an example, the biomarkers may be different or partially the same depending on the type of cancer.

The miRNAs of the present invention may be miR-1-3p, miR-139-3p, miR-124a-3p, miR-142-3p, miR-146b-5p, miR-15b-5p, miR-26b-5p, miR-25-3p, miR-34b-5p, miR-92a-3p, miR-126-3p, miR-148a-3p, miR-21-5P, miR-210-5p, miR-221-3p, miR-26a-5p, miR-29a-3p, miR-29b-3p, miR-29c-3p, miR-30a-5p, miR-34a-3p, miR-145-5p, miR-150-3P, miR-155-3P, miR-16b-5p, miR-18a-5p, miR-181b-5p, miR-223-3p, miR-26b-3p, miR-483-3p, miR-505-5p, miR-636-5p, miR-6761, miR-339-3P, miR-431, and miR-103-3p.

In addition, the miRNAs of the present invention may consist of SEQ ID NO: 1 to 36, respectively.

As an example, detection of the miRNAs of SEQ ID NOS: 1 to 36 may be performed using promers consisting of SEQ ID NOs: 39 to 74, respectively.

As an example, among the biomarkers, biomarkers specific to lung cancer may be miRNAs of miR-1-3p, miR-139-3p, miR-124a-3p, miR-142-3p, miR-146b-5p, miR-126-3p, miR-148a-3p, miR-21-5P, miR-210-5p, miR-221-3p, miR-26a-5p, miR-181b-5p, miR-223-3p, miR-26b-3p, miR-6761, and miR-339-3P.

In addition, the miRNAs of the present invention may consist of SEQ ID NOS: 1 to 4, SEQ ID NOS: 11 to 16, SEQ ID NOs: 27 to 29, SEQ ID NO: 33, and SEQ NO: 34, respectively.

As an example, detection of the miRNAs consisting of SEQ ID NOS: 1 to 4, SEQ ID NOS: 11 to 16, SEQ ID NOS: 27 to 29, SEQ ID NO: 33, and SEQ ID NO: 34 may be performed using promers consisting of SEQ ID NOs: 39 to 42, SEQ ID NOS: 49 to 62, SEQ ID NOs: 65 to 67, SEQ ID NO: 71, and SEQ ID NO: 72, respectively.

As an example, among the biomarkers, biomarkers specific to pancreatic cancer may be miRNAs of miR-29a-3p, miR-29b-3p, miR-29c-3p, miR-30a-5p, miR-34a-3p, miR-145-5p, miR-150-3P, miR-155-3P, miR-16b-5p, miR-181b-5p, miR-223-3p, miR-483-3p, miR-505-5p, miR-636-5p, miR-6761, and miR-339-3P.

In addition, the miRNAs of the present invention may consist of SEQ ID NOs: 17 to 25, SEQ ID NO: 27, SEQ ID NO: 28, and SEQ ID NOs: 30 to 34, respectively.

As an example, detection of the miRNAs consisting of SEQ ID NOS: 17 to 25, SEQ ID NO: 27, SEQ ID NO: 28, and SEQ ID NOs: 30 to 35 may be performed using promers consisting of SEQ ID NOS: 55 to 63, SEQ ID NO: 65, SEQ ID NO: 66, and SEQ ID NOS: 68 to 72, respectively.

As another specific example, among the biomarkers, biomarkers specific to colorectal cancer may be miRNAs of miR-1-3p, miR-15b-5p, miR-26b-5p, miR-25-3p, miR-34b-5p, miR-92a-3p, miR-18a-5p, miR-26b-3p, miR-483-3p, miR-505-5p, miR-636-5p, miR-6761, miR-339-3P, and miR-431.

In addition, the miRNAs of the present invention may consist of SEQ ID NO: 1, SEQ ID NOS: 6 to 10, SEQ ID NO: 26, and SEQ ID NO: 29 to 35, respectively.

As an example, detection of the miRNAs consisting of SEQ ID NO: 1, SEQ ID NOS: 6 to 10, SEQ ID NO: 26, and SEQ ID NOs: 29 to 35 may be performed using promers consisting of SEQ ID NO: 39, SEQ ID NOS: 44 to 48, SEQ ID NO: 64, and SEQ ID NOS: 67 to 73, respectively.

The biomarkers may include reference genes, and the reference genes may be miR-34b-5p, miR-29c-3p, miR-103-3p, and U43.

As an example, detection of the reference gene miR-34b-5p may be performed using a promer consisting of SEQ ID NO: 47, and detection of the reference gene miR-29c-3p may be performed using a promer consisting of SEQ ID NO: 57.

As an example, detection of the reference gene miR-103-3p may be performed using a promer consisting of SEQ ID NO: 74, and detection of the reference gene U43 may be performed using a promer consisting of SEQ ID NO: 75.

In addition, the reference genes included in the biomarkers may additionally include GAPDH. The GAPDH may serve as a reference gene for simultaneously identifying lung cancer, colorectal cancer, and pancreatic cancer, and is represented by SEQ ID NO: 38.

As an example, detection of the reference gene GAPDH may be performed using a promer consisting of SEQ ID NO: 76.

In the method according to one embodiment, when the multiple biomarkers are used as biomarkers for early diagnosis or prognostic prediction of multiple types of cancer (in particular, lung cancer, colorectal cancer, and pancreatic cancer), it is possible to increase the accuracy of diagnosis of multiple types of cancer (in particular, lung cancer, colorectal cancer, and pancreatic cancer), and provide information on the likelihood of metastasis of multiple types of cancer (in particular, lung cancer, colorectal cancer, and pancreatic cancer) (e.g., the potential for lung cancer, colon cancer, or pancreatic cancer to metastasize; the metastatic ability or invasive ability of lung cancer, colon cancer, or pancreatic cancer cells and/or tissue), and the potential of growth of multiple cancer (in particular, lung cancer, colorectal cancer, and pancreatic cancer) tissues (e.g., an increase in the number of lung cancer, colon cancer, or pancreatic cancer cells that developed in the lung, large intestine, or pancreas) and/or in the size of tumor tissue), and/or predict the prognosis (e.g., survival rate) of patients with multiple types of cancer (in particular, lung cancer, colorectal cancer, and pancreatic cancer), thereby providing important information for strategies for treatment of multiple types of cancer (particularly lung cancer, colorectal cancer, and pancreatic cancer).

In another aspect, the present invention provides a composition or a kit for early diagnosis or prognostic prediction of multiple types of cancer.

The composition or kit comprises multiple biomarkers for diagnosis or prognostic prediction of multiple types of cancer, which are as described above.

In addition to the multiple biomarkers, the composition or kit comprises promers for detecting the biomarkers for early diagnosis or prognostic prediction of multiple types of cancer.

The promer is a single nucleic acid developed by the present inventors, and refers to a single nucleic acid that binds to the cDNA of a miRNA and may be used as both a probe and a primer. For the promer, reference may be made to Korean Patent No. 10-1901749 and Korean Patent No. 10-1991440.

Specifically, the promer is characterized in that it has a structure of X-Y-Z and in that one or more detectable markers are attached at both ends or inside of the promer. In this case, the positions of the detectable markers attached are not limited to specific sites, and may be any positions at which the detectable markers are separated when the region Y is cleaved by a specific enzyme.

X in the structure of X-Y-Z is a DNA consisting of 1 to 60, preferably 1 to 30, more preferably 2 to 30, even more preferably 3 to 30, even more preferably 4 to 30, even more preferably 5 to 30, even more preferably 6 to 30, even more preferably 7 to 30, even more preferably 8 to 30, even more preferably 10 to 30 nucleotides. It is a structure that acts as a primer when Y is cleaved and Y and Z are separated from the template. If X comprises more than 60 nucleotides, non-specific reactions may occur during real-time detection of nucleic acids or proteins.

The region Y consists of one or two RNAs, and when the region Y has been bound to the template, the region Y is cleaved by a ribonuclease (RNase) and separated from the template. Examples of the ribonuclease include RNase II, RNase III, RNase IV, RNaseH, and RNase T2.

Therefore, the composition or kit may further comprise a ribonuclease (RNase) for cleaving the region Y of the promer.

The region Z consists of 1 to 3, preferably 2 or 3 DNAs, and is separated from the template along with the region Y when the region Y is cleaved.

The detectable marker that is used in the present invention may be either a fluorophore that binds to the promer through a covalent or non-covalent bond, or a fluorescent pair composed of the fluorophore and a quencher.

The fluorophore may be, for example, any one selected from the group consisting of Cy® 3, Cy® 5, Cy® 5.5, Bodipy™, Alexa™ 488, Alexa™ 532, Alexa™ 546, Alexa™ 568, Alexa™ 594, Alexa™ 660, rhodamine, TAMRA™, FAM™, FITC, Fluor™ X, ROX™, Texas Red™, Orange green 488X, Orange green 514X, HEX™, TET∩, JOE™, Oyster® 556, Oyster® 645, Bodipy™ 630/650, Bodipy™ 650/665, Calfluor™ Orange 546, Calfluor™ red 610, Quasar® 670, and biotin, without being necessarily limited thereto. In addition, the quencher may be, for example, any one selected from the group consisting of DDQ-1, Dabcyl, Eclipse™, 6-TAMRA™, BHQ®-1, BHQ®-2, BHQ®-3, lowa Black® RQ-Sp, QSY™-7, QSY™-2, and MGBNFQ, without being necessarily limited thereto.

In the present invention, when the fluorescent pair is used as the detectable marker, the fluorophore and the quencher may be located in the region X or Z or the region Y, without being limited thereto. As an example, the fluorophore may be located in X, and the quencher may be located in Y or Z.

The composition or kit may further comprise reagents necessary for synthesis reactions for cDNAs of multiple biomarkers.

The reagents necessary for synthesis reactions include, for example, suitable amounts of reverse transcriptase, an RT primer such as oligo (dT) 18 primer or random primer, nuclease-free water, 4× DN master mix, and the like.

The composition or kit may further comprise reagents necessary for DNA amplification reaction.

The reagents for amplification reaction include, for example, suitable amounts of DNA polymerase (e. g., thermostable DNA polymerase obtained from Thermus aquaticus (Taq), Thermus thermophiles (Tth), Thermus filiformis, Thermis flavus, Thermococcus litoralis or Pyrococcus furiosus (Pfu)), DNA polymerase cofactor (Mg²⁺), buffer solution, dNTPs (dATP, dCTP, dGTP and dTTP), and water (dH₂O). In addition, the buffer solution includes suitable amounts of Triton X-100, dimethylsulfoxide (DMSO), Tween 20, nonidet P40, PEG 6000, formamide, and bovine serum albumin (BSA), without being limited thereto.

The composition or kit may further comprise a 96-well plate.

In still another aspect, the present invention provides a method of providing information necessary for early diagnosis or prognostic prediction of multiple types of cancer using multiple biomarkers or a composition containing the same.

In a more specific aspect, the present invention provides a method for early diagnosis or prognostic prediction of multiple types of cancer (or a method of providing information for diagnosis or prognostic prediction) comprising a step of detecting multiple biomarkers in a biological sample isolated from a subject.

As an example, the method of providing information for early diagnosis or prognostic prediction of multiple types of cancer may further comprise steps of: comparing and classifying detection information of the multiple biomarkers detected in the detection step; and early diagnosing or predicting prognosis of multiple types of cancer based on the detection information of multiple biomarkers classified in the above step.

As an example, the method of providing information for early diagnosis or prognostic prediction of multiple types of cancer may further comprise a step of inputting detection information of the multiple biomarkers detected in the detection step into a trained artificial intelligence model and analyzing the input information.

In the above method, the step of detecting biomarkers may be a step of detecting the presence or absence of the biomarkers in the sample, or measuring the expression levels (e.g., Ct values) of the biomarkers, or performing both. In one example, the step of detecting biomarkers may be performed by polymerase chain reaction (PCR), reverse transcription-polymerase chain reaction (RT-PCR), real-time polymerase chain reaction (real-time PCR), or quantitative reverse transcription-polymerase chain reaction (RT-qPCR). Preferably, the step of detecting biomarkers is performed by quantitative reverse transcription-polymerase chain reaction (RT-qPCR). In one example, the step of detecting biomarkers may be performed using promers, and the promers are as described above.

As used herein, the term “biological sample” or “sample” may refer to blood and liquid samples of biological origin. The biological sample or sample may be one isolated from a subject (e.g., a subject with a cancer disease and/or a subject who is likely to develop lung cancer, colorectal cancer, or pancreatic cancer).

As an example, the biological sample may be whole blood, plasma, serum, or the like.

As another example, the biological sample may be obtained from an animal, such as a mammal, or a human. For example, the biological sample may be one isolated from a patient with lung cancer, colorectal cancer, or pancreatic cancer, or from a person who is likely to develop lung cancer, colorectal cancer, or pancreatic cancer. The biological sample may be pretreated prior to the detection step. For example, the biological sample may be subjected to pretreatment steps such as homogenization, filtration, distillation, extraction, concentration, inactivation of interfering components, addition of reagents, etc.

The subject (individual) may be an individual suspected of having multiple types of cancer, particularly lung cancer, colorectal cancer, or pancreatic cancer, and/or an individual having multiple types of cancer, particularly lung cancer, colorectal cancer, or pancreatic cancer.

As an example, the subject and/or the biological sample isolated from the subject may be one in which the multiple biomarkers described above are detected.

The method of providing information for diagnosis or prognostic prediction of multiple types of cancer (particularly lung cancer, colorectal cancer, or pancreatic cancer) according to one example is capable of detecting, as the biomarkers for diagnosis or prognostic prediction of multiple types cancer (particularly of lung cancer, colorectal cancer, or pancreatic cancer), 20, 21, 22, 23, 24, 25, 26, 27, 28, 29, 30, 31, 32, 33, 34, 35, or 36 biomarkers selected from the group consisting of miR-1-3p, miR-139-3p, miR-124a-3p, miR-142-3p, miR-146b-5p, miR-15b-5p, miR-26b-5p, miR-25-3p, miR-34b-5p, miR-92a-3p, miR-126-3p, miR-148a-3p, miR-21-5P, miR-210-5p, miR-221-3p, miR-26a-5p, miR-29a-3p, miR-29b-3p, miR-29c-3p, miR-30a-5p, miR-34a-3p, miR-145-5p, miR-150-3p, miR-155-3p, miR-16b-5p, miR-18a-5p, miR-181b-5p, miR-223-3p, miR-26b-3p, miR-483-3p, miR-505-5p, miR-636-5p, miR-6761, miR-339-3p, miR-431, and miR-103-3p.

In addition, the biomarkers may further comprise U43 and GAPDH, which may be detected.

The method of providing information for diagnosis or prognostic prediction of lung cancer according to one specific example is capable of detecting 6, 7, 8, 9, 10, 11, 12, 13, 14, 15, or 16 biomarkers selected from the group consisting of miR-1-3p, miR-139-3p, miR-124a-3p, miR-142-3p, miR-146b-5p, miR-126-3p, miR-148a-3p, miR-21-5P, miR-210-5p, miR-221-3p, miR-26a-5p, miR-181b-5p, miR-223-3p, miR-26b-3p, miR-6761, and miR-339-3P. In this case, it is possible to increase the accuracy of diagnosis of lung cancer, and predict the likelihood of metastasis of lung cancer (the likelihood of metastasis and/or infiltration of lung cancer (e.g., lung cancer cells)), and the likelihood of growth of lung cancer tissue (an increase in the number of cancer cells present in the lung and/or the size of tumor tissue present in the lung), and/or the prognosis (e.g., survival rate) of lung cancer patients, thereby providing important information for strategies for treatment of lung cancer.

The method of providing information for diagnosis or prognostic prediction of pancreatic cancer according to another specific example is capable of detecting 6, 7, 8, 9, 10, 11, 12, 13, 14, 15, or 16 biomarkers selected from the group consisting of miR-29a-3p, miR-29b-3p, miR-29c-3p, miR-30a-5p, miR-34a-3p, miR-145-5p, miR-150-3P, miR-155-3P, miR-16b-5p, miR-181b-5p, miR-223-3p, miR-483-3p, miR-505-5p, miR-636-5p, miR-6761, and miR-339-3P. In this case, it is possible to increase the accuracy of diagnosis of pancreatic cancer, and predict the likelihood of metastasis of pancreatic cancer (the likelihood of metastasis and/or infiltration of pancreatic cancer (e.g., pancreatic cancer cells)), and the likelihood of growth of pancreatic cancer tissue (an increase in the number of cancer cells present in the pancreas and/or the size of tumor tissue present in the pancreas), and/or the prognosis (e.g., survival rate) of pancreatic cancer patients, thereby providing important information for strategies for treatment of pancreatic cancer.

The method of providing information for diagnosis or prognostic prediction of colorectal cancer according to another specific example is capable of detecting 6, 7, 8, 9, 10, 11, 12, 13, or 14 biomarkers selected from the group consisting of miR-1-3p, miR-15b-5p, miR-26b-5p, miR-25-3p, miR-34b-5p, miR-92a-3p, miR-18a-5p, miR-26b-3p, miR-483-3p, miR-505-5p, miR-636-5p, miR-6761, miR-339-3P, and miR-431. In this case, it is possible to increase the accuracy of diagnosis of colorectal cancer, and predict the likelihood of metastasis of colorectal cancer (the likelihood of metastasis and/or infiltration of colorectal cancer (e.g., colorectal cancer cells)), and the likelihood of growth of colorectal cancer tissue (an increase in the number of cancer cells present in the large intestine and/or the size of tumor tissue present in the large intestine), and/or the prognosis (e. g., survival rate) of pancreatic cancer patients, thereby providing important information for strategies for treatment of colorectal cancer.

The method of providing information for early diagnosis or prognostic prediction of multiple types of cancer (preferably lung cancer, colon cancer, or pancreatic cancer) may further comprise steps of: comparing and classifying detection information of the multiple biomarkers detected in the detection step; and determining the type of cancer of the sample based on the detection information of multiple biomarkers classified in the above step.

The detection information of the multiple biomarkers may be the expression level of each of the multiple biomarkers. In addition, the detection information may be information obtained from a 96-well plate biochip capable of simultaneous detection of multiple biomarkers (e.g., 36 types of miRNA). For example, the expression level may be a Ct value determined by quantitative reverse transcription-PCR (RT-qPCR).

The comparison may be made by comparing the detection information of multiple biomarkers measured from the sample with previously measured detection information of multiple biomarkers in normal people. The previously measured detection information of multiple biomarkers in normal people may be the expression levels of 36 types of miRNA according to the present invention and the expression levels of reference genes for each of lung cancer, colorectal cancer, and pancreatic cancer among the 36 types of miRNA.

As an example, the detection information of multiple biomarkers in normal people may be the expression levels of miR-1-3p, miR-139-3p, miR-124a-3p, miR-142-3p, miR-146b-5p, miR-15b-5p, miR-26b-5p, miR-25-3p, miR-34b-5p, miR-92a-3p, miR-126-3p, miR-148a-3p, miR-21-5P, miR-210-5p, miR-221-3p, miR-26a-5p, miR-29a-3p, miR-29b-3p, miR-29c-3p, miR-30a-5p, miR-34a-3p, miR-145-5p, miR-150-3P, miR-155-3P, miR-16b-5p, miR-18a-5p, miR-181b-5p, miR-223-3p, miR-26b-3p, miR-483-3p, miR-505-5p, miR-636-5p, miR-6761, miR-339-3P, miR-431, and miR-103-3p in normal people.

As an example, the previously measured detection information in normal people may be the Ct values of multiple biomarkers, determined by RT-PCR and listed in Table 1 below.

TABLE 1

| miRNA | Ct value | miRNA | Ct value | miRNA | Ct value |
|---|---|---|---|---|---|
| miR-34b-5p | 35.8 | miR-26a-5p | 24.8 | miR-6761 | 33.1 |
| miR-124a-3p | 32.5 | miR-148a-3p | 26.9 | miR-339-3p | 30.9 |
| miR-1-3p | 29.7 | miR-30a-5p | 25.7 | miR-126-3p | 25.8 |
| miR-146b-5p | 30.2 | miR-210-5p | 32.3 | miR-34a-3p | 30.0 |
| miR-26b-5p | 24.4 | miR-150-3p | 32.6 | miR-29a-3p | 25.2 |
| miR-92a-3p | 23.6 | miR-155-3p | 35.6 | miR-145-5p | 28.3 |
| miR-142-3p | 25.1 | miR-16b-5p | 21.2 | miR-18a-5p | 26.9 |
| miR-139-3p | 31.1 | miR-181b-5p | 32.3 | miR-26b-3p | 32.3 |
| miR-25-3p | 26.0 | miR-223-3p | 24.3 | miR-21-5p | 24.2 |
| miR-15b-5p | 25.1 | miR-483-3p | 28.7 | miR-221-3p | 25.4 |
| miR-29b-3p | 25.1 | miR-505-5p | 33.6 | miR-431 | 32.3 |
| miR-29c-3p | 24.2 | miR-636-5p | 33.3 | miR-103-3p | 24.2 |

The classification may mean classifying the results of comparing the detection information of multiple biomarkers detected from the sample with the detection information of multiple biomarkers in normal people. As an example, the detection information may be the expression levels of multiple biomarkers. As an example, classifying the results of comparison may be performed by classifying the detection information (e.g., expression levels) into four groups: increase by 2-fold or more, increase by less than 2-fold, decrease by less than ½-fold, and decrease by more than ½-fold, compared to the detection information of multiple biomarkers in normal people.

Determining the type of cancer of the sample based on the classified detection information of multiple biomarkers may comprise determining that a case, in which at least 6 of biomarkers (reference genes) specific to each cancer type have the same detection information (e.g., expression levels) as the criteria shown in Table 13 below while at least 20 of the multiple biomarkers have the same detection information (e.g., expression levels) as the criteria shown in Table 13 below, is a corresponding cancer type.

As an example, the biomarkers specific to lung cancer may be miR-1-3p, miR-139-3p, miR-124a-3p, miR-142-3p, miR-146b-5p, miR-126-3p, miR-148a-3p, miR-21-5P, miR-210-5p, miR-221-3p, miR-26a-5p, miR-181b-5p, miR-223-3p, miR-26b-3p, miR-6761, and miR-339-3P.

As an example, the biomarkers specific to colorectal cancer may be miR-1-3p, miR-15b-5p, miR-26b-5p, miR-25-3p, miR-34b-5p, miR-92a-3p, miR-18a-5p, miR-26b-3p, miR-483-3p, miR-505-5p, miR-636-5p, miR-6761, miR-339-3P, and miR-431.

As an example, the biomarkers specific to pancreatic cancer may be miR-29a-3p, miR-29b-3p, miR-29c-3p, miR-30a-5p, miR-34a-3p, miR-145-5p, miR-150-3P, miR-155-3P, miR-16b-5p, miR-181b-5p, miR-223-3p, miR-483-3p, miR-505-5p, miR-636-5p, miR-6761, and miR-339-3P.

Determining the type of cancer based on the classified detection information of multiple biomarkers may comprise determining that a case, in which increases in detection information (e.g., expression levels) of at least 5 biomarkers selected from the group consisting of miR-139-3p, miR-29c-3p, miR-148a-3p, miR-30a-5p, miR-210-5p, miR-150-3p, miR-181b-5p, miR-34a-3p, miR-29a-3p, miR-145-5p, miR-26b-3p, miR-221-3p, and miR-431 are 2-fold or more while at least 20 of the multiple biomarkers have the same detection information (e.g., expression levels) as the criteria shown in Table 13 below, is pancreatic cancer among multiple types of cancers.

TABLE 13

| | Lung cancer | Colorectal cancer | Pancreatic cancer |
|---|---|---|---|
| miR-34b-5p | + | + | ++++ |
| miR-124a-3p | + | + | + |
| miR-1-3p | ++ | + | ++++ |
| miR-146b-5p | ++++ | ++ | ++++ |
| miR-26b-5p | ++++ | ++++ | ++++ |
| miR-92a-3p | ++++ | ++++ | ++ |
| miR-142-3p | ++++ | ++++ | ++++ |
| miR-139-3p | + | + | ++++ |
| miR-25-3p | ++++ | ++++ | ++++ |
| miR-15b-5p | ++++ | ++++ | ++++ |
| miR-29b-3p | ++++ | ++++ | ++++ |
| miR-29c-3p | | ++ | ++++ |
| miR-26a-5p | ++++ | +++ | ++++ |
| miR-148a-3p | ++ | + | ++++ |
| miR-30a-5p | ++ | + | ++++ |
| miR-210-5p | + | + | ++++ |
| miR-150-3p | + | + | ++++ |
| miR-155-3p | + | ++ | + |
| miR-16b-5p | ++++ | ++++ | +++ |
| miR-181b-5p | | + | ++++ |
| miR-223-3p | +++ | +++ | ++++ |
| miR-483-3p | + | + | + |
| miR-505-5p | ++++ | ++++ | ++++ |
| miR-636-5p | ++ | +++ | ++ |
| miR-6761 | + | + | +++ |
| miR-339-3p | +++ | ++ | ++++ |
| miR-126-3p | ++++ | | ++++ |
| miR-34a-3p | + | + | ++++ |
| miR-29a-3p | + | + | ++++ |
| miR-145-5p | + | + | ++++ |
| miR-18a-5p | ++++ | ++++ | ++++ |
| miR-26b-3p | ++ | + | ++++ |
| miR-21-5p | +++ | + | ++++ |
| miR-221-3p | + | + | ++++ |
| miR-431 | | + | ++++ |
| miR-103-3p | ++++ | ++++ | ++++ |
| GAPDH | ++++ | + | ++++ |

In Table 13 above, ++++ denotes a case in which the relative increase in the expression level of the biomarker relative to the expression level of the biomarker in normal people is 2-fold or more, +++ denotes a case in which the relative increase in the expression level of the biomarker relative to the expression level of the biomarker in normal people is less than 2-fold, ++ denotes a case in which the relative decrease in the expression level of the biomarker relative to the expression level of the biomarker in normal people is less than ½-fold, and + denotes a case in which the relative decrease in the expression level of the biomarker relative to the expression level of the biomarker in normal people is ½-fold or more. Meanwhile, in Table 13, those without symbols denote a case in which the expression level of the biomarker is almost equal to the expression level of the biomarker in normal people.

As an example, the method of providing information for early diagnosis or prognostic prediction of multiple types of cancer (preferably lung cancer, colorectal cancer, or pancreatic cancer) according to one example may further comprise a step of inputting the measured detection information of the multiple biomarkers into a trained artificial intelligence model and analyzing the input information.

The detection information of the multiple biomarkers may be the expression level for each of the multiple biomarkers. In addition, the detection information may be information obtained from a 96-well plate biochip capable of simultaneous detection of multiple biomarkers (e.g., 36 types of miRNA). For example, the expression level may be a Ct value determined by real-time PCR.

The trained artificial intelligence model is a model obtained by classifying detection information of previously detected multiple biomarkers into a training dataset, a validation dataset, and a test dataset, modeling a Deep Neural Network (DNN) algorithm, and testing the modeled DNN algorithm.

In addition, the trained artificial intelligence model may be one trained by setting detection information of multiple biomarkers in normal people and a patient group as input values.

The detection information of the multiple biomarkers in normal people and the patient group may be the expression levels of miR-1-3p, miR-139-3p, miR-124a-3p, miR-142-3p, miR-146b-5p, miR-15b-5p, miR-26b-5p, miR-25-3p, miR-34b-5p, miR-92a-3p, miR-126-3p, miR-148a-3p, miR-21-5P, miR-210-5p, miR-221-3p, miR-26a-5p, miR-29a-3p, miR-29b-3p, miR-29c-3p, miR-30a-5p, miR-34a-3p, miR-145-5p, miR-150-3P, miR-155-3P, miR-16b-5p, miR-18a-5p, miR-181b-5p, miR-223-3p, miR-26b-3p, miR-483-3p, miR-505-5p, miR-636-5p, miR-6761, miR-339-3P, miR-431, and miR-103-3p in normal people and the patient group.

The expression levels of the multiple biomarkers may be the Ct values of the multiple biomarkers detected.

As an example, the detection information of multiple biomarkers in normal people may be the Ct values of the multiple biomarkers, determined by real-time PCR and shown in Table 1 above.

The detection information of the multiple biomarkers may be the Ct value of each of the multiple biomarkers detected in a 96-well plate biochip capable of simultaneous detection of multiple biomarkers (e.g., 36 types of miRNA).

The 96-well plate biochip is a biochip in which reagents for detecting 36 types of miRNA are distributed in 96 wells and from which the expression levels may be measured.

In addition, the trained artificial intelligence model may be a model into which detection information of multiple biomarkers measured in the sample is input and which outputs, as an output value, determination of the type of cancer of the sample.

The output value may be a single or multi-labeled value for multiple types of cancer and normal. As an example, the output value may be a single or multi-labeled output value for lung cancer, colon cancer, pancreatic cancer, and normal.

The artificial intelligence algorithm used in the artificial intelligence model may be a deep neural network (DNN), which is an artificial neural network comprising a plurality of hidden layers between at least one input layer and an output layer. In addition, it may be a recurrent neural network (RNN), a restricted Boltzmann machine (RBM), or the like.

In this case, single-layer perceptron in deep learning is the most basic model of artificial neural network and comprises an input layer that receives and connects external input values, and an output layer that processes the input values and outputs the results. Multi-layer perceptron is one of the theories of artificial neural network and is also called back-propagation network or multi-Layer feedforward network. Basically, multi-layer perceptron consists of an input layer, a hidden layer, and an output layer, and each of the layers has a number of neurons (nodes). Here, the back-propagation algorithm gives each node of the input layer a random weight according to the input pattern, and this signal is transmitted to the hidden layer and reaches the output layer to compare the output value with the target value. In order to reduce the error derived from the comparison, the connection strength is adjusted and backward progress is made. The back-propagation algorithm is used to reduce the error by repeating this process.

Artificial neural networks and deep learning have a structure that derives output values through an input layer, a hidden layer, and an output layer, and nerves are transmitted through the connection strength between the layers. By setting parameters such as appropriate hidden layer, learning rate, loss function, activation function, momentum, maximum number of learning processes, and number of learning process repetitions, it is possible to prevent problems such as gradient loss and overfitting and to construct a model with good performance. In one embodiment of the present invention, hyper-parameters may be used to find suitable parameters, but the use of other methods is not excluded.

Hyper-parameters are used to find optimized parameters by learning and trying various set parameters. Here, there are ReLU (Rectified Linear Unit) and TanH (Hyperbolic Tangent) emd as activation functions to compensate for gradient loss, learning convergence speed, overfitting, etc. When the weight is positive, the ReLU function appears as it is, and when the weight is positive, gradient loss does not occur even if there are multiple hidden layers. The TanH function has a form similar to Sigmoid but can be said to be an expanded form. Since TanH function is expanded to −1 instead of 0, the output range is wide, making faster convergence possible.

As an example, using a trained artificial intelligence model, was possible to early diagnose multiple types of cancer with an accuracy of about 90% or more from the expression levels of input multiple biomarkers and to predict the prognosis of multiple types of cancer.

According to the present invention, simultaneous early diagnosis, and prognostic diagnosis of various types of cancer in a non-invasive manner are possible, and such diagnosis and prognostic prediction are characterized by excellent specificity and sensitivity and high accuracy. Therefore, the present invention will be helpful in the treatment and prevention of cancer through early diagnosis of cancer.

In addition, using an artificial intelligence model, it is possible to simultaneously diagnose and predict prognosis of various types of cancer with high accuracy in a simple, fast and non-invasive manner.

### BRIEF DESCRIPTION OF THE DRAWINGS

FIG. 1 shows the results of detection of reference genes as miRNAs extracted from the blood of colorectal cancer patients according to one example of the present invention.

FIG. 2 shows the results of detection of reference genes as miRNAs extracted from the blood of pancreatic cancer patients according to one example of the present invention.

FIG. 3 shows the results of screening of reference genes for colorectal cancer and pancreatic cancer using the RefFinder program according to one example of the present invention.

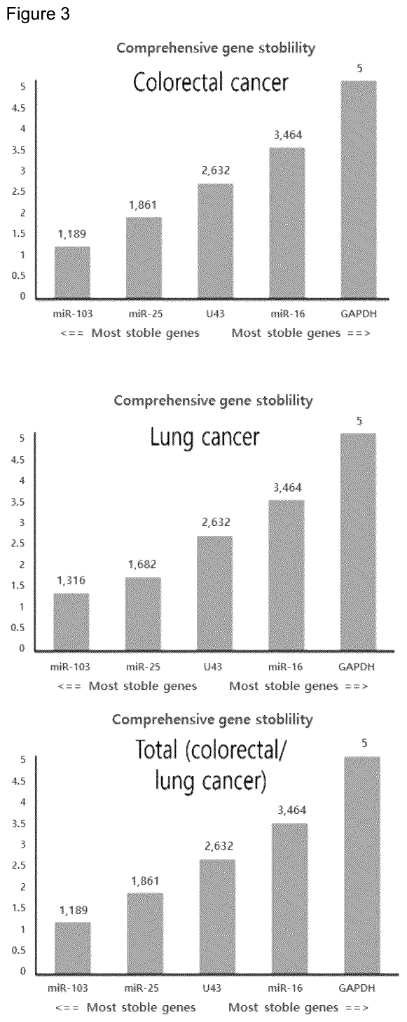

FIG. 4 shows the results of confirming whether analysis of 37 types of miRNA targets for each cancer type isolated from blood according to one example of the present invention is possible.

FIG. 5 shows the results of comparing the expression patterns of lung cancer miR-29b-3p, pancreatic cancer miR-223-3p, colorectal cancer miR-92a-3p, and internal control (reference gene) miR-103-3p as miRNA targets for each cancer type, isolated from blood, between patients and normal people according to one example of the present invention.

FIG. 6 shows the configuration of a 96-well plate biochip prepared according to one example of the present invention.

FIG. 7 shows the mean and standard deviation of Ct values for 37 types of miRNA targets for each cancer type, isolated from blood according to one example of the present invention.

FIG. 8 shows the expression levels of miRNAs as numerical values based on the average Ct values of 37 types of miRNA targets for each cancer type, isolated from blood, relative to the Ct values of miRNAs in normal people, according to one example of the present invention. The X-axis lists miRNAs in the order shown in Table 13, and the Y-axis shows the relative expression levels relative to those in normal people. Blue indicates lung cancer, orange indicates colon cancer, and gray indicates pancreatic cancer.

FIG. 9 shows a matrix for each of a training set, a validation set, a test set, and a mixture thereof to construct an artificial intelligence model according to one example of the present invention.

FIG. 10 shows the distribution of cancer diagnosis results derived from a trained artificial intelligence model based on a Ct value for each of 20 normal people and 50 cancer patients, derived by a biochip system according to one example of the present invention.

FIG. 11 shows the specificity and sensitivity of cancer diagnosis results derived from a trained artificial intelligence model based on a Ct value for each of 20 normal people and 50 cancer patients, derived by a biochip system according to one example of the present invention.

FIG. 12 shows the accuracy of cancer diagnosis derived from a trained artificial intelligence model based on a Ct value for each of 20 normal people and 50 cancer patients, derived by a biochip system according to one example of the present invention.

### DETAILED DESCRIPTION

Hereinafter, the present invention will be described in detail with reference to examples to aid understanding of the present invention. However, the examples according to the present invention may be modified into various other forms, and the scope of the present invention should not be construed as being limited to the following examples. The examples of the present invention are provided to more completely explain the present invention to those skilled in the art.

### Example 1: Specification of Reference Genes for Diagnosis of Lung Cancer, Colorectal Cancer, and Pancreatic Cancer

In order to increase the precision and accuracy of diagnosis of lung cancer, colon cancer, and pancreatic cancer, candidate reference genes were selected with reference to papers (Non-Patent Documents [1] and [2]) to normalize each result value to reference genes. The selected genes are shown in Table 2 below.

TABLE 2

| Reference | | | | |
|---|---|---|---|---|
| gene | RNA Class | NCBI reference | Definition | Size(bp) |
| GAPDH | mRNA | NM_002046.7 | Homo sapiens glyceraldehyde- | 1285 |
| | | | 3-phosphate dehydrogenate | |
| | | | (GAPDH), transcript variant 1, | |
| | | | mRNA | |
| SNORD48; | Samll nucleolar | NR_002745 | Homo sapiens small nucleolar | 63 |
| RUN48; U48 | RNA(snoRNA) | | RNA, C/D box 48 (SNORD48), | |
| | | | small nucleolar RNA | |
| SNORD44; | Samll nucleolar | NR_002750.2 | Homo sapiens small nucleolar | 62 |
| RUN44; U44 | RNA(snoRNA) | | RNA, C/D box 44 (SNORD44), | |
| | | | small nucleolar RNA | |
| SNORD43; | Samll nucleolar | NR_002439 | Homo sapiens small nucleolar | 62 |
| RUN43; U43 | RNA(snoRNA) | | RNA, C/D box 43 (SNORD43), | |
| | | | small nucleolar RNA | |
| RUN6B; U6 | Samll nucleolar | NR_002752.2 | Homo sapiens RNA, U6 small | 107 |
| | RNA(snoRNA) | | nuclear 2 (RNU6-2), small | |
| | | | nuclear RNA | |
| hsa-miR-16- | microRNA(miRNA) | MIMAT0000069 | Homo sapiens microRNA 16 | 22 |
| 5p | | | (MIR16), microRNA. | |
| hsa-miR-25- | microRNA(miRNA) | MIMAT0000081 | Homo sapiens microRNA 25 | 22 |
| 3p | | | (MIR25), microRNA. | |
| hsa-miR- | microRNA(miRNA) | MIMAT0000440 | Homo sapiens microRNA 191 | 23 |
| 191-5p | | | (MIR191), microRNA. | |
| hsa-miR- | microRNA(miRNA) | MIMAT0000101 | Homo sapiens microRNA 103a | 23 |
| 103a-3p | | | (MIR103a), microRNA. | |
| hsa-miR-93- | microRNA(miRNA) | MIMAT0000093 | Homo sapiens microRNA 93 | 23 |
| 5p | | | (MIR93), microRNA. | |

Among the candidate reference genes in Table 2 above, U48, U44, U6, miR-191, and miR-93, which are reference genes with low detection levels in RNA extracted from blood, were excluded, and GAPDH, U43, and miR-103, miR-25, and miR-16 genes were first selected. Thereafter, an experiment for detecting five reference genes (GAPDH, U43, miR-103, miR-25, and miR-16) from RNA extracted from the blood of colon cancer patients and pancreatic cancer patients was performed to target genes that could be used as reference genes.

Detection of five reference genes was performed using the promers, RT primers, and reverse primers described in Tables 3 to 6 below. The promer is a single nucleic acid developed by the present inventors, has a structure composed of DNA, RNA, and DNA, and is a single nucleic acid that can be used as both a primer and a probe (see Korean Patent No. 10-1901749). This experiment was repeated twice, and each mean value at the time of detection was expressed as a Ct value. For each Ct value, the mean value for each target was expressed, and then the SD value was expressed to confirm suitability for the mean value.

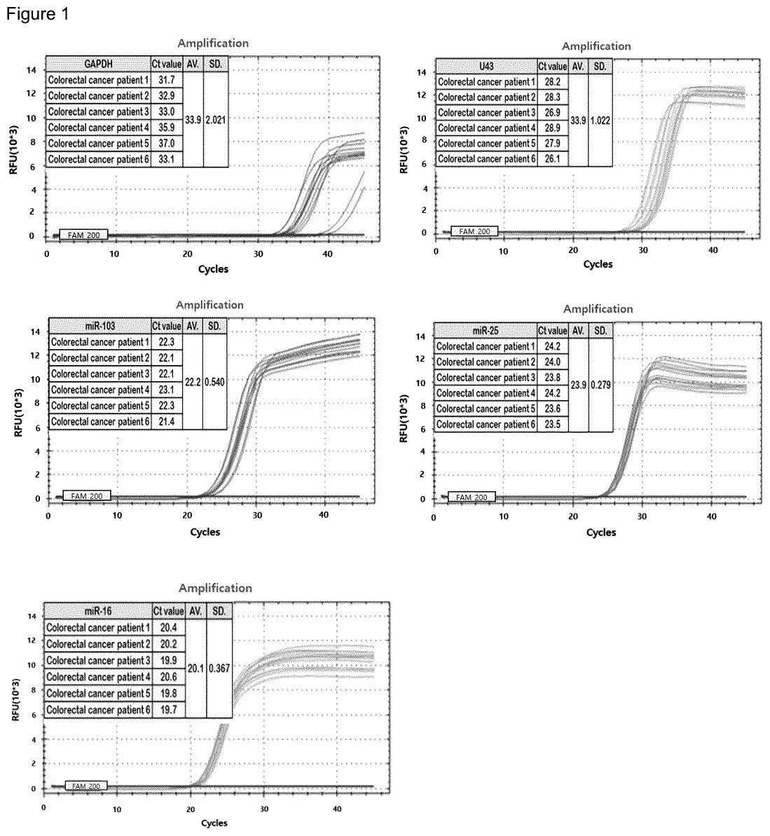

As a result, as shown in FIG. 1, the SD values for colorectal cancer patients were 0.279 for miR-25, 0.367 for miR-16, 0.540 for miR-103, 1.022 for U43, and 2.021 for GAPDH. Therefore, miR-25, which showed the lowest SD value, was considered the most stable reference gene.

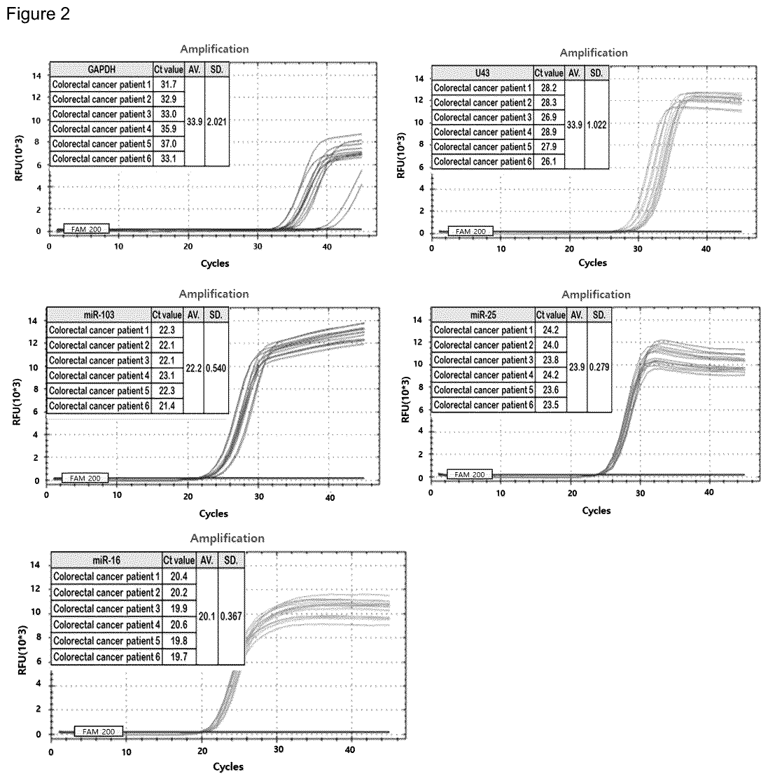

In addition, as shown in FIG. 2, the SD values for pancreatic cancer patients were 0.735 for U43, 1.392 for miR-25, 1.445 for miR-103, 1.487 for miR-16, and 2.989 for GAPDH. Therefore, U43, which showed the lowest SD value, was considered the most stable reference gene.

For more accurate analysis, the analysis was performed using the RefFinder program. As a result, as can be seen in FIIG. 3, it was found that the most stable reference gene was miR-103 in colorectal cancer patient samples, miR-25 in pancreatic cancer patient samples, and miR-103 in both colorectal cancer patient samples and pancreatic cancer patient samples.

From the above experimental results, it could be seen that the most stable types of miRNA for colorectal cancer patients and pancreatic cancer patients are different. That is, in the case of miRNA, the type of stable target may vary depending on the type of sample. Therefore, it was determined that not only miR-103 but also miR-25, miR-16, and GAPDH should be used as reference genes, rather than using one reference gene, in order to simultaneously identify multiple types of cancer.

### Example 2: Identification of miRNA Analysis Ability for each Cancer Type in Blood and Selection of miRNAS

Because miRNAs are complexly involved in metabolism, cell proliferation, apoptosis, developmental timing, etc., it is difficult to diagnose a single cancer type with a single miRNA, and there may be more than one type of cancer in which a single type of miRNA is involved. Therefore, cancer should be diagnosed by analyzing the expression patterns of several types of miRNAs.

From the RNA extracted from the blood of normal people and patients, miRNA targets for three cancer types, lung cancer, pancreatic cancer, and colon cancer, were selected, and 35 miRNA targets and 2 internal controls (reference genes) as shown in Tables 3 and 4 below were selected as significant miRNA targets. Among these selected miRNAs, miR-25-3p and miR-16b-5p are targets related to colorectal cancer and pancreatic cancer, respectively, but may be used as internal controls (reference genes) for cancers other than colorectal cancer and cancers other than pancreatic cancer, respectively.

Of the total 37 targets, 32 targets were detectable using FAM dye, and miR-34b-5p, miR-124a-3p, miR-1-3p, miR-146b-5p, and miR-26b-5p were detectable by multiplex real-time PCR using Texas Red dye. Thereby, it was possible to detect the 37 targets in one experiment using a 96-well plate biochip. Promers, RT primers, and reverse primers for detecting the 32 targets are shown in Tables 5 and 6 below. In the promers in Tables 5 and 6, RNA is written with a lowercase letter followed by an uppercase letter, and for example, there are rG, rC, rU, and rA in the promer sequences.

TABLE 3

| | | | | | | SEQ |
|---|---|---|---|---|---|---|
| | Lung | Pancreatic | Colorectal | Reference | | ID |
| miRNA | cancer | cancer | cancer | gene | Sequence | NO. |
| miR-1-3p | ○ | | ○ | | ugg aau gua aag aag | 1 |
| | | | | | uau gua u | |
| miR-139-3p | ○ | | | | ugg aga cgc ggc ccu | 2 |
| | | | | | guu gga gu | |
| miR-124a- | ○ | | | | uaa ggc acg cgg uga | 3 |
| 3p | | | | | aug cca a | |
| miR-142-3p | ○ | | | | ugu agu guu ucc uac | 4 |
| | | | | | uuu aug ga | |
| miR-146b- | ○ | | | | uga gaa cug aau ucc | 5 |
| 5p | | | | | aua ggc ug | |
| miR-15b-5p | | | ○ | | uag cag cac auc aug | 6 |
| | | | | | guu uac a | |
| miR-26b-5p | | | ○ | | uuc aag uaa uuc agg | 7 |
| | | | | | aua ggu | |
| miR-25-3p | | | ○ | | cau ugc acu ugu cuc | 8 |
| | | | | | ggu cug a | |
| miR-34b-5p | | | ○ | ○ | uag gca gug uca uua | 9 |
| | | | | | gcu gau ug | |
| miR-92a-3p | | | ○ | | uau ugc acu ugu ccc | 10 |
| | | | | | ggc cug u | |
| miR-126-3p | ○ | | | | ucg uac cgu gag uaa | 11 |
| | | | | | uaa ugc g | |
| miR-148a- | | | | | uca gug cac uac aga | 12 |
| 3p | | | | | acu uug u | |
| miR-21-5P | ○ | | | | uag cuu auc aga cug | 13 |
| | | | | | aug uug a | |
| miR-210-5p | ○ | | | | agc ccc ugc cca ccg | 14 |
| | | | | | cac acu g | |
| miR-221-3p | ○ | | | | agc uac auu guc ugc | 15 |
| | | | | | ugg guu uc | |
| miR-26a-5p | ○ | | | | uuc aag uaa ucc agg | 16 |
| | | | | | aua ggc u | |
| miR-29a-3p | | ○ | | | uag cac cau cug aaa | 17 |
| | | | | | ucg guu a | |
| miR-29b-3p | | ○ | | | uag cac cau uug aaa | 18 |
| | | | | | uca gug uu | |
| miR-29c-3p | | ○ | | ○ | uag cac cau uug aaa | 19 |
| | | | | | ucg guu a | |
| miR-30a-5p | | ○ | | | ugu aaa cau ccu cga | 20 |
| | | | | | cug gaa g | |
| miR-34a-3p | | ○ | | | ugg cag ugu cuu agc | 21 |
| | | | | | ugg uug u | |
| miR-145-5p | | ○ | | | guc cag uuu ucc cag | 22 |
| | | | | | gaa ucc cu | |

TABLE 4

| | | | | | | SEQ |
|---|---|---|---|---|---|---|
| | Lung | Pancreatic | Colorectal | Reference | | ID |
| miRNA | cancer | cancer | cancer | gene | Sequence | NO. |
| miR-150-3P | | ○ | | | cug gua cag gcc ugg | 23 |
| | | | | | ggg aca g | |
| miR-155-3P | | ○ | | | cuc cua cau auu agc | 24 |
| | | | | | auu aac a | |
| miR-16b-5p | | ○ | | | uag cag cac gua aau | 25 |
| | | | | | auu ggc g | |
| miR-18a-5p | | | ○ | | uaa ggu gca ucu agu | 26 |
| | | | | | gca gau ag | |
| miR-181b- | ○ | ○ | | | aac auu cau ugc ugu | 27 |
| 5p | | | | | cgg ugg gu | |
| miR-223-3p | ○ | ○ | | | ugu cag uuu guc aaa | 28 |
| | | | | | uac ccc a | |
| miR-26b-3p | ○ | | ○ | | ccu guu cuc cau uac | 29 |
| | | | | | uug gcu | |
| miR-483-3p | | ○ | ○ | | uca cuc cuc ucc ucc | 30 |
| | | | | | cgu cuu | |
| miR-505-5p | | | ○ | | ggg agc cag gaa gua | 31 |
| | | | | | uug aug u | |
| miR-636-5p | | ○ | O | | ugu gcu ugc ucg ucc | 32 |
| | | | | | cgc ccg ca | |
| miR-6761 | ○ | ○ | ○ | | ucu gag aga gcu cga | 33 |
| | | | | | ugg cag | |
| miR-339-3P | ○ | ○ | ○ | | uga gcg ccu cga cga | 34 |
| | | | | | cag agc cg | |
| miR-431 | | | ○ | | ugu cuu gca ggc cgu | 35 |
| | | | | | cau gca | |
| miR-103-3p | | | | ○ | agc agc auu gua cag | 36 |
| | | | | | ggc uau ga | |
| U43 | | | | ○ | cacagatgat | 37 |
| | | | | | gaacttattg | |
| | | | | | acgggcggac | |
| | | | | | agaaactgtg | |
| | | | | | tgctgattgt | |
| | | | | | cacgttctga tt | |
| GAPDH | | | | | <see SEQ ID NO: 38 | 38 |
| | | | | | of the sequence | |
| | | | | | listing> | |

TABLE 5

| | | SEQ ID |
|---|---|---|
| Promer | Sequence | NO. |
| miR-1-3p promer | 5′-/Texas Red/ GCC ACT GGA ATG TAA AGA AGT | 39 |
| | ArUG T/3IABKFQ/-3′ | |
| miR-139-3p promer | 5′-/56-FAM/TTA TTA GAC GCG GCC CTrG TT/ | 40 |
| | 3IABKFQ/-3′ | |
| miR-124a-3p promer | 5′-/5TexRd-XN/AAT TA AGG CAC GCG GTG ArAT | 41 |
| | GC/3IABKFQ/-3′ | |
| miR-142-3p promer | 5′-/56-FAM/GCC CTG TAG TGT TTC CTA CTT TrAT | 42 |
| | G/3IABKFQ/-3′ | |
| miR-146b-5p promer | 5′-/Texas Red/GCC ACT GAG AAC TGA ATT CCA | 43 |
| | TrAG G/3IABKFQ/-3′ | |
| miR-15b-5p promer | 5′-/56-FAM/GCG TAT AGC AGC ACA TCA TGG | 44 |
| | rUTT/3IABKFQ/-3′ | |
| miR-26b-5p promer | 5′-/Texas Red/CGCT TCA AGT AAT TCA GGA | 45 |
| | TAGrGTA/3IABKFQ/-3′ | |
| miR-25-3p promer | 5′-/56-FAM/ACA TTG CAC TTG TCT CGG rUCT | 46 |
| | /3IABKFQ/-3′ | |
| miR-34b-5p promer | 5′-/5TexRd-XN/ CTAG GCA GTG TCA TTA GCT GrAT | 47 |
| | T/3IABKFQ/-3′ | |
| miR-92a-3p promer | 5′-/56-FAM/GCC TAT ATT GCA CTT GTC | 48 |
| | rCCG/3IABKFQ/-3′ | |
| miR-126-3p promer | 5′-/56-FAM/GG TCG TAC CGT GAG TAA TAA TrGC G/ | 49 |
| | 3IABKFQ/-3′ | |
| miR-148a-3p promer | 5′-/56-FAM/ ACC TCA GTG CAC TAC AGA ACrU | 50 |
| | TT/3IABKFQ/-3′ | |
| miR-21-5P promer | 5′-/56-FAM/GCC AC T AGC TTA TCA GAC TGA TrGT | 51 |
| | T/3IABKFQ/-3′ | |
| miR-210-5p promer | 5′-/56-FAM/ATGC CCA CCG CAC rACT/3IABKFQ/-3′ | 52 |
| miR-221-3p promer | 5′-/56-FAM/TAG CTA CAT TGT CTG CTG GrGT | 53 |
| | T/3IABKFQ/-3′ | |
| miR-26a-5p promer | 5′-/56-FAM/ CCA CT TCA AGT AAT CCA GGA | 54 |
| | TAGrGCT/3IABKFQ/-3′ | |
| miR-29a-3p promer | 5′-/56-FAM/GAG TAG CAC CAT CTG AAA TCrG | 55 |
| | GT/3IABKFQ/-3′ | |
| miR-29b-3p promer | 5′-/56-FAM/CCC TAG CAC CAT TTG AAA TCrA | 56 |
| | GT/3IABKFQ/-3′ | |
| miR-29c-3p promer | 5′-/56-FAM/GAG TAG CAC CAT TTG AAA TCrG | 57 |
| | GT/3IABKFQ/-3′ | |
| miR-30a-5p promer | 5′-/56-FAM/GAC TGT AAA CAT CCT CGA CTG | 58 |
| | rGAA/3IABKFQ/-3′ | |
| miR-34a-3p promer | 5′-/56-FAM/TTG GCA GTG TCT TAG CTG | 59 |
| | rGTT/3IABkFQ/-3′ | |
| miR-145-5p promer | 5′-/56-FAM/TTG TCC AGT TTT CCC AGG AArU | 60 |
| | CC/3IABKFQ/-3′ | |

TABLE 6

| | | SEQ ID |
|---|---|---|
| Promer or primer | Sequence | NO. |
| miR-150-3P promer | 5′-/56-FAM/TAT CTG GTA CA(A) GCC TGrG | 61 |
| | GG/3IABKFQ/-3′ | |
| miR-155-3P promer | 5′-/56-FAM/CC ACC TCC TAC ATA TTA GCA TTrA | 62 |
| | ACA/3IABKFQ/-3′ | |
| miR-16b-5p promer | 5′-/56-FAM/GG TAG CAG CAC GTA (G)AT ATT GrGC | 63 |
| | G/3IABKFQ/-3′ | |
| miR-18a-5p promer | 5′-/56-FAM/GG GTG TAA GGT GCA TCT ArGT | 64 |
| | G/3IABKFQ/-3′ | |
| miR-181b-5p promer | 5′-/56-FAM/TCA TTG CTG TCG GTG rGGT/3IABKFQ/- | 65 |
| | 3′ | |
| miR-223-3p promer | 5′-/56-FAM/TTG TCA GTT TGT CAA ATA CCrC | 66 |
| | CA/3IABKFQ/-3′ | |
| miR-26b-3p promer | 5′-/56-FAM/ C CAC CCC TGT TCT CCA TTA CrUT | 67 |
| | G/3IABKFQ/-3′ | |
| miR-483-3p promer | 5′-/56-FAM/TCA CTC CTC TCC TCC CrGT | 68 |
| | C/3IABKFQ/-3′ | |
| miR-505-5p promer | 5′-/56-FAM/GGG AGC CAG GAA GTA TTG | 69 |
| | rATG/3IABKFQ/-3′ | |
| miR-636-5p promer | 5′-/56-FAM/ATG TGC TTG CTC GTC rCCG/ | 70 |
| | 3IABKFQ/-3′ | |
| miR-6761 promer | 5′-/56-FAM/TCT CCT ACG CTG CTC TCT | 71 |
| | rCAC/3IABKFQ/-3′ | |
| miR-339-3P promer | 5′-/56-FAM/TAT AGC GCC TCG ACG ACrA | 72 |
| | GA/3IABKFQ/-3′ | |
| miR-431 promer | 5′-/56-FAM/TTA TGT CTT GCA GGC CGT | 73 |
| | rCAT/3IABKFQ/-3′ | |
| miR-103-3p promer | /56-FAM/AGC AGC ATT GTA CAG rGGC/3IABKFQ/ | 74 |
| U43 promer | 5′-/56-FAM/AAC TGT GTG CTG ATT GTC | 75 |
| | rACG/3IABKFQ/-3′ | |
| GAPDH promer | /56-FAM/GAG CCG CAC CTT GTrC AT/3IABKFQ/ | 76 |
| RT primer | 5′-GTT GAT TTT TTC TTT TTC AAC TTT TTT TTT | 77 |
| | TTT TTT TTT-3′ | |
| Reverse primer | 5′-GTT GAT TTT TTC TTT TTC AAC TTT TTT TTT | 78 |
| | TTT TTT TTT-3′ | |

### Example 2: Verification of Lowest Analyte Concentration in Plasma

For the 35 miRNA targets and internal controls (reference genes) related to lung cancer, pancreatic cancer, and colon cancer, shown in Tables 3 and 4 above, 400 ng of RNA extracted from plasma isolated from normal blood samples was synthesized into cDNA through poly A tailing & RT PCR, the analytical ability of each target was examined by real-time PCR.

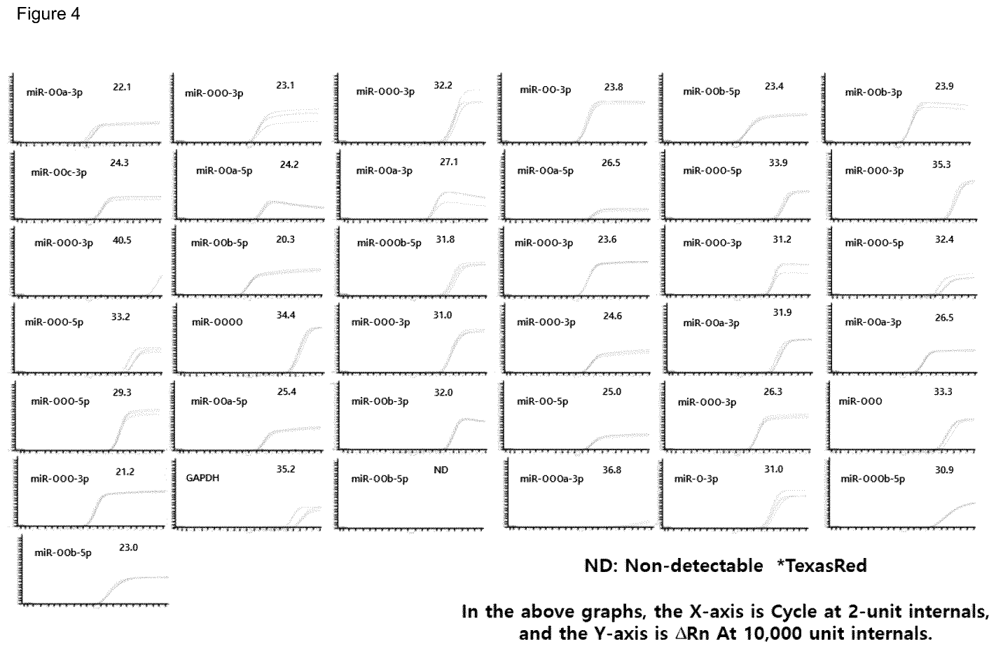

The values obtained in this experiment are the experimental results in triplicate, and the mean values of Ct at the time of detection are shown with a graph (see FIG. 4). As can be seen in FIG. 4, the Ct values were very different for each target, with Ct 20.3 for miR-16b-5p and ND (non-detected) for miR-34b-5p. This is because the types and amounts of miRNAs present in plasma isolated from blood were different, and thereby, it could be confirmed that analysis of miRNA detection patterns for each cancer type was possible.

### Example 3: Detection and Analysis of miRNAs from Patient and Control Plasma Samples

### 3-1. Comparative Analysis of Significant Difference in miRNAs Extracted from Blood between Cancer Types

First, plasma was isolated from each of blood samples of patients with three types of cancer (lung cancer, pancreatic cancer, and colon cancer) and blood samples of normal people, and the amounts of RNAs extracted from the plasma samples were quantified using a UV spectrophotometer (NanoDrop, Thermo Scientific) and compared with each other. The results are shown in Table 7 below.

TABLE 7

| | | Lung | Pancreatic | Colorectal |
|---|---|---|---|---|
| | Normal | cancer | cancer | cancer |
| | people | patients | patients | patients |
| | N = 127 | N = 102 | N = 50 | N = 57 |
| Mean | 14.03 | 15.85 | 14.18 | 20.82 |
| concentration | | | | |
| value (ng/ul) | | | | |
| SD | 8.42 | 4.84 | 6.57 | 6.97 |
| CV (%) | 60 | 31 | 45 | 33 |

As can be seen in Table 7 above, as a result of examining the mean concentrations, SD values, and CV (%) values of the RNAs extracted from the blood samples of normal people, lung cancer patients, pancreatic cancer patients, and colon cancer patients, it was found that the colon cancer patients showed the highest mean concentration value, but the CV (%) value for the entire data was in the range of 31 to 60, suggesting that there was no significant difference in the amount of extracted RNA between cancer types.

### 3-2. Detection of Cancer-Related miRNAs from RNA Extracted from Blood for Each Cancer Type

Using equal amounts (10 ng) of RNA extracted from plasma isolated from blood for each cancer type as shown in Table 7 above, CDNA was synthesized by poly A tailing & RT PCR. Using the cDNA, target miRNAs for each cancer type were detected by real-time PCR.

Using lung cancer miR-29b-3p, pancreatic cancer miR-223-3p, colorectal cancer miR-92a-3p, and internal control (reference gene) miR-103-3p as miRNA targets for each cancer type, the expression level of each miRNA in the RNA extracted from plasma isolated from the blood of lung cancer, pancreatic cancer and colorectal cancer patients and from the blood of normal people was analyzed using the promer for each target (see Tables 5 and 6).

As a result, the Ct values of the internal control (reference gene) in lung cancer patients, pancreatic cancer patients, colon cancer patients, and normal people were 21.6, 21.7, 21.6, and 21.9, respectively, showing similar values of SD 0.12.

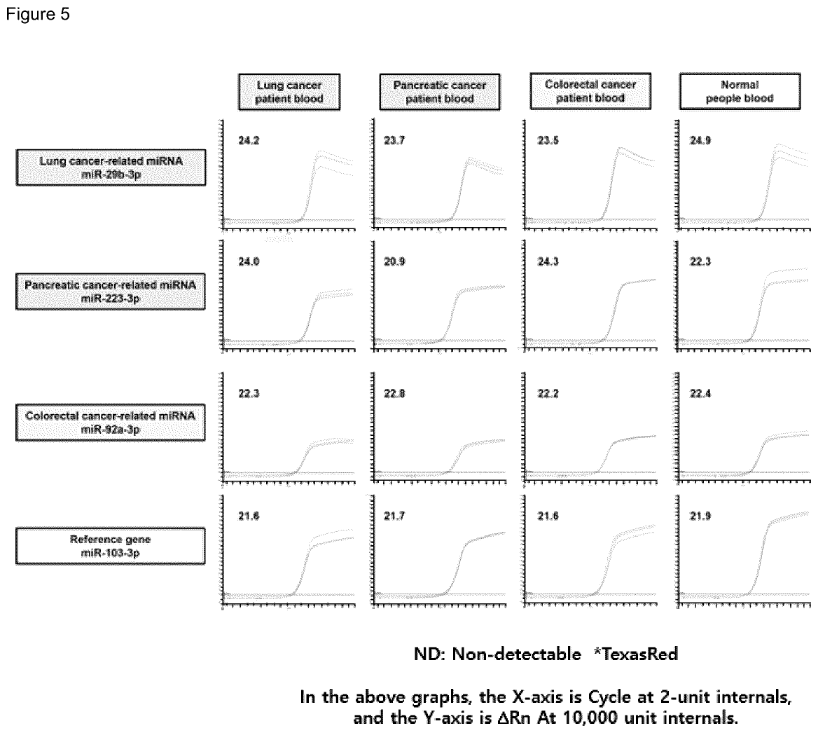

miR-29b-3p, a lung cancer-related miRNA, showed Ct values of 24.2, 23.7, 23.5, and 24.9 in lung cancer, pancreatic cancer, colon cancer, and normal, respectively (see FIG. 5). When these values were normalized to the internal control Ct value and then compared with the value in normal people, it was found that miR-29b-3p was up-regulated in the cancer patients and the values thereof were higher in the order of colon cancer>pancreas cancer>lung cancer.

miR-223-3p, a pancreatic cancer-related miRNA, showed Ct values of 24, 20.9, 24.3, and 22.3 in lung cancer, pancreatic cancer, colon cancer, and normal, respectively (see FIG. 5). When these values were normalized to the internal control Ct value and then compared with the value in normal people, it was found that miR-223-3p was up-regulated in the pancreatic cancer patients and down-regulated in the lung cancer patients and the colorectal cancer patients.

MiR-92a-3p, a colorectal cancer-related miRNA, showed Ct values of 22.3, 22.8, 22.2, and 22.4 in lung cancer, pancreatic cancer, colon cancer, and normal, respectively (see FIG. 5). When these values were normalized to the internal control Ct value and then compared with the value in normal people, it was found that MiR-92a-3p was down-regulated in the cancer patients and the values thereof were higher in the order of pancreatic cancer>lung cancer>colorectal cancer, but the difference in values was not significant.

As a result of the above experiment, it was found that the difference in miRNAs between the blood samples was greatest for miR-223-3p, a pancreatic cancer-related miRNA, and there was no significant difference in the above experiment results for the remaining targets. Therefore, it was found that it is not easy to diagnose a cancer type with only one target miRNA.

### Example 4: Construction of Diagnostic System using Biochip

Using a 96-well plate biochip in which promers for the 35 miRNAs and internal controls s (reference genes) finally selected for each cancer type were distributed, a diagnostic method using miRNAs related to each cancer type was established.

Specifically, RNA was extracted from 200 μl of plasma isolated from one blood sample using the Maxwell® RSC miRNA Plasma and Serum kit (Promega). The extracted RNA was adjusted to the same concentration of 5 ng/μl and then synthesized into cDNA by poly A tailing & RT PCR using the poly A tailing & RT PCR composition shown in Table 8 below and the poly A tailing & RT PCR conditions shown in Table 9 below.

TABLE 8

| No. | Component | 1 rxn (μl) |
|---|---|---|
| 1 | Poly(A) tailing & RT master mix (2X) | 100 |
| 2 | RT primer | 10 |
| 3 | Poly(A) tailing enzyme | 5 |
| 4 | Reverse transcriptase | 1.35 |
| 5 | Nuclease-free water | 63.65 |
| 6 | miRNA template/NTC | 20 |
| | Total reaction volume | 200 |

TABLE 9

| Step | Temperature | Time |
|---|---|---|
| Incubation | 40° C. | 60 min |
| Inactivation | 95° C. | 10 min |
| Cooling | 10° C. | 10 min |

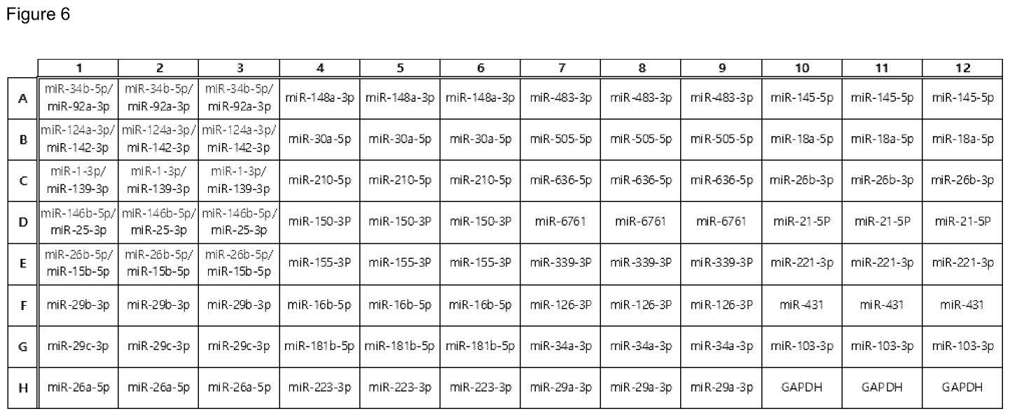

A 96-well plate biochip in which a real-time PCR mixture prepared according to the real-time PCR composition shown in Table 10 below using the synthesized cDNA and 37 kinds of promers (5 μl for each promer; the concentrations of the promers are different) were distributed (see FIG. 6) was mounted in the epMotion 5070 (Eppendorf), and then 10 μl of the real-time PCR mixture was dispensed into each well of the 96-well plate biochip.

TABLE 10

| No. | Component | 100 rxn (μl) |
|---|---|---|
| 1 | 3X real-time PCR master mix | 500 |
| 2 | Reverse primer | 100 |
| 3 | Nuclease free water | 200 |
| 4 | RT product (cDNA) | 200 |
| | Total reaction volume | 1000 |

After completion of the dispensing process, the 96-well plate biochip was sealed, and then the mixture centrifuged and then subjected to real-time PCR under the PCR conditions shown in Table 11 below. Based on the real-time PCR results, the Ct value was analyzed under the followed set values using the following set values: threshold FAM: 200, 000, Texas Red: 150, 000, and cut off: 35.

TABLE 11

| Step | Temperature | Time | |
|---|---|---|---|
| Pre-denaturation | 95° C. | 5 | min |
| Denaturation | 95° C. | 10 | sec |
| Annealing & extension | 65° C. | 1 | min |

(Data acquisition)

### Example 5: Results of Diagnosis of each Cancer Type by Analysis of Multiple miRNAS

Diagnosis of each cancer type was performed on clinical samples using the diagnostic system of Example 4.

Specifically, plasma was separated from blood samples collected from 87 lung cancer pancreatic cancer patients, 51 colorectal cancer patients, and 83 normal people, and RNA was extracted from the plasma samples using the Maxwell® RSC miRNA Plasma and Serum kit (Promega). CDNA was synthesized from 100 ng of the extracted RNA by poly A tailing & RT PCR and then subjected to real-time PCR using the 96-well plate biochip system of Example 4.

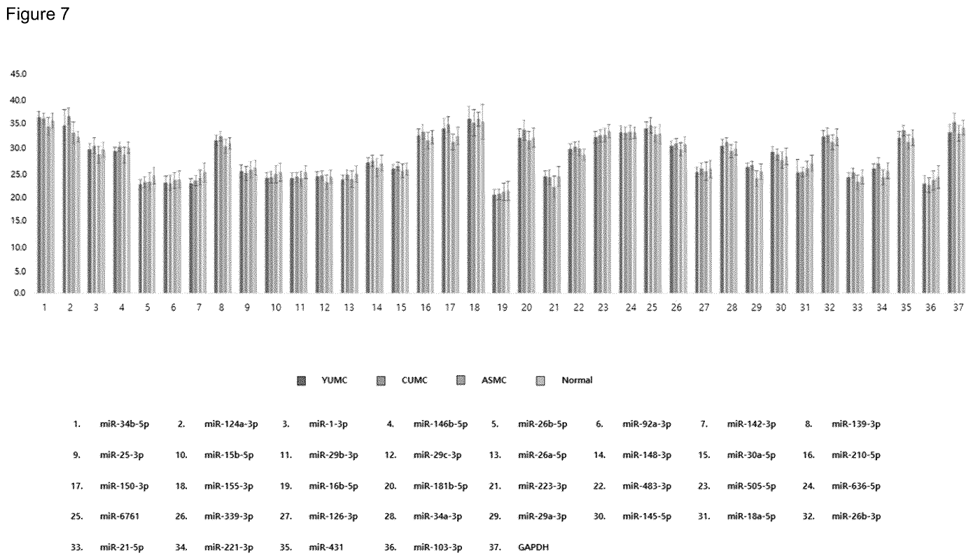

As a result of the experiment, the mean Ct value and SD value for each of the 37 targets for each cancer type are shown in Table 12 below and FIG. 7.

TABLE 12

| | | | Colorectal | | Pancreatic | | | |
|---|---|---|---|---|---|---|---|---|
| | Lung cancer | | cancer | | cancer | | Normal | |
| | patients | | patients | | patients | | people | |
| | N = 87 | | N = 51 | | N = 51 | | N = 83 | |
| | Ct | | Ct | | Ct | | Ct | |
| | Mean | SD | Mean | SD | Mean | SD | Mean | SD |
| miR-34b-5p | 36.4 | 1.34 | 36.2 | 1.20 | 34.6 | 1.78 | 35.8 | 1.63 |
| miR-124a-3p | 34.8 | 3.24 | 36.7 | 1.69 | 33.3 | 2.19 | 32.5 | 1.15 |
| miR-1-3p | 29.8 | 1.36 | 30.6 | 1.63 | 28.8 | 1.78 | 29.7 | 1.56 |
| miR-146b-5p | 29.4 | 0.93 | 30.4 | 0.94 | 28.6 | 1.65 | 30.2 | 1.19 |
| miR-26b-5p | 22.6 | 0.99 | 23.0 | 1.11 | 23.1 | 1.85 | 24.4 | 1.77 |
| miR-92a-3p | 22.9 | 1.36 | 22.8 | 1.52 | 23.4 | 1.67 | 23.6 | 1.74 |
| miR-142-3p | 22.8 | 0.95 | 23.4 | 1.03 | 24.0 | 1.55 | 25.1 | 1.97 |
| miR-139-3p | 31.7 | 1.05 | 32.6 | 1.04 | 30.4 | 1.40 | 31.1 | 1.22 |
| miR-25-3p | 25.3 | 1.30 | 24.9 | 1.46 | 25.6 | 1.70 | 26.0 | 1.62 |
| miR-15b-5p | 23.9 | 1.16 | 24.1 | 1.25 | 24.6 | 1.79 | 25.1 | 1.85 |
| miR-29b-3p | 23.8 | 1.17 | 24.1 | 0.97 | 23.8 | 1.54 | 25.1 | 1.38 |
| miR-29c-3p | 24.2 | 0.95 | 24.4 | 1.09 | 23.1 | 1.41 | 24.2 | 1.42 |
| miR-26a-5p | 23.6 | 0.95 | 24.5 | 1.06 | 23.7 | 1.74 | 24.8 | 1.66 |
| miR-148a-3p | 27.1 | 1.01 | 27.5 | 1.12 | 26.0 | 1.80 | 26.9 | 1.65 |
| miR-30a-5p | 25.8 | 0.93 | 26.3 | 0.92 | 25.4 | 1.43 | 25.7 | 1.30 |
| miR-210-5p | 32.7 | 1.41 | 33.4 | 1.61 | 31.6 | 1.78 | 32.3 | 1.39 |
| miR-150-3p | 34.1 | 2.04 | 34.9 | 1.66 | 31.3 | 1.64 | 32.6 | 1.83 |
| miR-155-3p | 36.1 | 2.53 | 35.3 | 2.68 | 36.1 | 1.43 | 35.6 | 3.58 |
| miR-16b-5p | 20.4 | 1.13 | 20.5 | 1.14 | 21.1 | 1.83 | 21.2 | 1.98 |
| miR-181b-5p | 32.3 | 1.73 | 33.8 | 2.14 | 31.7 | 1.76 | 32.3 | 1.96 |
| miR-223-3p | 24.2 | 1.22 | 24.0 | 1.63 | 22.1 | 2.19 | 24.3 | 2.10 |
| miR-483-3p | 29.9 | 1.08 | 30.4 | 0.99 | 30.1 | 1.51 | 28.7 | 1.14 |
| miR-505-5p | 32.3 | 1.16 | 32.7 | 1.22 | 32.7 | 1.36 | 33.6 | 1.43 |
| miR-636-5p | 33.4 | 1.36 | 33.2 | 1.27 | 33.4 | 1.44 | 33.3 | 1.22 |
| miR-6761 | 34.1 | 1.40 | 34.8 | 1.58 | 32.8 | 1.60 | 33.1 | 1.96 |
| miR-339-3p | 30.6 | 1.01 | 31.0 | 1.06 | 29.8 | 1.40 | 30.9 | 1.62 |
| miR-126-3p | 25.2 | 1.00 | 25.8 | 1.21 | 25.3 | 1.87 | 25.8 | 1.85 |
| miR-34a-3p | 30.5 | 1.36 | 31.4 | 0.89 | 29.5 | 1.41 | 30.0 | 1.41 |
| miR-29a-3p | 26.2 | 0.83 | 26.5 | 0.89 | 23.8 | 1.57 | 25.2 | 1.61 |
| miR-145-5p | 29.2 | 1.32 | 28.8 | 1.22 | 27.6 | 1.66 | 28.3 | 1.75 |
| miR-18a-5p | 25.1 | 2.69 | 25.1 | 1.09 | 25.9 | 1.52 | 26.9 | 1.66 |
| miR-26b-3p | 32.4 | 1.30 | 32.8 | 1.45 | 31.3 | 1.59 | 32.3 | 1.72 |
| miR-21-5p | 24.1 | 0.92 | 25.0 | 0.95 | 23.1 | 1.44 | 24.2 | 1.53 |
| miR-221-3p | 25.8 | 1.06 | 26.9 | 1.09 | 24.1 | 1.47 | 25.4 | 1.69 |
| miR-431 | 32.3 | 1.21 | 33.7 | 1.10 | 31.3 | 1.47 | 32.3 | 1.69 |
| miR-103-3p | 22.7 | 1.66 | 22.4 | 1.58 | 23.4 | 2.03 | 24.2 | 2.38 |
| GAPDH | 33.3 | 1.74 | 35.4 | 1.84 | 33.1 | 1.60 | 34.3 | 1.49 |

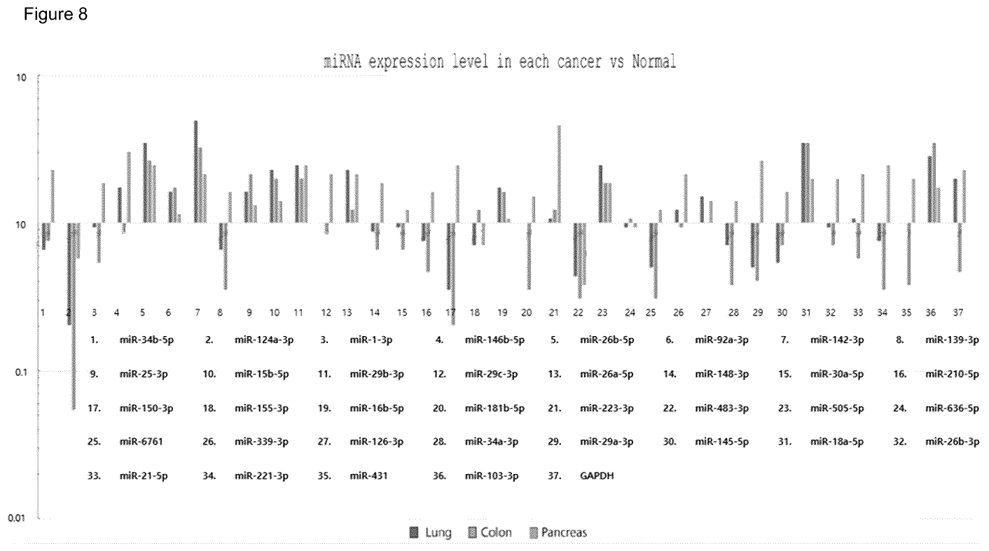

The Ct values for lung cancer, colon cancer, and pancreatic cancer were compared with the measured Ct values for normal people, and the expression levels of miRNAs are shown in FIG. 8. In this case, the relative expression level of the miRNA is 2 to the power of n, where n is (Ct value of miRNA in normal people—Ct value of miRNA in the sample). Referring to FIG. 8, the lung cancer, colon cancer, and pancreatic cancer patients showed different patterns in expression levels compared to those in normal people. These differences in expression levels are shown in Table 13 below. In Table 13, ++++ denotes a case in which the relative increase in the expression level of miRNA relative to that in normal people is 2-fold or more, and +++ denotes a case in which the relative increase in the expression level of miRNA relative to that in normal people is less than 2-fold. In addition, ++ denotes a case in which the relative decrease in the expression level of miRNA relative to that in normal people is less than ½-fold, and + denotes a case in which the relative decrease in the expression level of miRNA relative to that in normal people is ½-fold or more. Meanwhile, in Table 13, those without symbols denote a case in which the expression level of miRNA almost equal to that in normal people.

TABLE 13

| | Lung cancer | Colorectal cancer | Pancreatic cancer |
|---|---|---|---|
| miR-34b-5p | + | + | ++++ |
| miR-124a-3p | + | + | + |
| miR-1-3p | ++ | + | ++++ |
| miR-146b-5p | ++++ | ++ | ++++ |
| miR-26b-5p | ++++ | ++++ | ++++ |
| miR-92a-3p | ++++ | ++++ | ++ |
| miR-142-3p | ++++ | ++++ | ++++ |
| miR-139-3p | + | + | ++++ |
| miR-25-3p | ++++ | ++++ | ++++ |
| miR-15b-5p | ++++ | ++++ | ++++ |
| miR-29b-3p | ++++ | ++++ | ++++ |
| miR-29c-3p | | ++ | ++++ |
| miR-26a-5p | ++++ | +++ | ++++ |
| miR-148a-3p | ++ | + | ++++ |
| miR-30a-5p | ++ | + | ++++ |
| miR-210-5p | + | + | ++++ |
| miR-150-3p | + | + | ++++ |
| miR-155-3p | + | ++ | + |
| miR-16b-5p | ++++ | ++++ | +++ |
| miR-181b-5p | | + | ++++ |
| miR-223-3p | +++ | +++ | ++++ |
| miR-483-3p | + | + | + |
| miR-505-5p | ++++ | ++++ | ++++ |
| miR-636-5p | ++ | +++ | ++ |
| miR-6761 | + | + | +++ |
| miR-339-3p | +++ | ++ | ++++ |
| miR-126-3p | ++++ | | ++++ |
| miR-34a-3p | + | + | ++++ |
| miR-29a-3p | + | + | ++++ |
| miR-145-5p | + | + | ++++ |
| miR-18a-5p | ++++ | ++++ | ++++ |
| miR-26b-3p | ++ | + | ++++ |
| miR-21-5p | +++ | + | ++++ |
| miR-221-3p | + | + | ++++ |
| miR-431 | | + | ++++ |
| miR-103-3p | ++++ | ++++ | ++++ |
| GAPDH | ++++ | + | ++++ |

Ct values for 36 miRNAs were measured for 20 lung cancer patients, 20 colon cancer patients, 20 pancreatic cancer patients, and 20 normal people by the above-described method, and the expression levels relative to those in normal people were determined and compared with the relative expression levels shown in Table 13 above. As a result, it was shown that the relative expression levels of at least 20 miRNAs among 36 miRNAs were similar to those shown in Table 13. In addition, among the 36 miRNAs, the relative expression levels of at least 6 miRNAs specific to lung cancer, colon cancer, and pancreatic cancer (see the miRNAs marked with O in Tables 2 and 3) were found to be similar to those shown in Table 13. In addition, as can be seen in Table 13, the expression levels of miR-139-3p, miR-29c-3p, miR-148a-3p, miR-30a-5p, miR-210-5p, miR-150-3p, miR-181b-5p, miR-34a-3p, miR-29a-3p, miR-145-5p, miR-26b-3p, miR-221-3p, and miR-431 decreased in lung cancer and colorectal cancer, but increased by 2-fold or more in pancreatic cancer, and thus the pancreatic cancer patients tested showed a two-fold or more increase in the expression levels of 5 or more of the 13 miRNAs. According to this method, 2 out of 20 people with lung cancer were misdiagnosed as normal and 18 people were correctly diagnosed with lung cancer; 1 out of 20 people with pancreatic cancer was misdiagnosed with colorectal cancer and 19 people were correctly diagnosed with pancreatic cancer; and 3 out of 20 people with colorectal cancer were misdiagnosed as normal, and 17 people were correctly diagnosed with colorectal cancer, indicating that this method showed a high accuracy of 90.0% in the ability to distinguish between cancer types.

Therefore, the Ct values of the 36 miRNAs for a sample may be measured and then compared with the Ct values in normal people (see Table 12) to determine the relative expression levels of the 36 miRNAs. A case, in which a case, in which the relative expression levels of at least 6 miRNAs among miRNAs specific to each cancer type are similar to those shown in Table 13 while the relative expression levels of at least 20 of the miRNAs are similar to the relative expression levels shown in Table 13 above, may be determined to be a corresponding cancer. In addition, a case, in which the expression levels of five or more miRNAs among 13 miRNAs, including miR-139-3p, miR-29c-3p, miR-148a-3p, miR-30a-5p, miR-210-5p, miR-150-3p, miR-181b-5p, miR-34a-3p, miR-29a-3p, miR-145-5p, miR-26b-3p, miR-221-3p, and miR-431 increased by two-fold or more compared to those in normal people, may be determined to be pancreatic cancer.

### Example 6: Analysis of Clinical Effectiveness of miRNAS

The number of oncogenes or tumor suppressors targeted by miRNAs has not yet been clearly identified. This suggests that miRNAs have the ability to target multiple genes as they can promote targeting of multiple compensatory pathways. However, specific miRNA targets include not only oncogenes and tumor suppressors but also numerous targets not related to cancer. In addition, multifaceted investigation of miRNAs is required because they are likely to have unknown targets that may adversely affect genes by targeting normal cellular homeostasis genes, especially at non-physiological concentrations. Accordingly, the present inventors attempted to effectively diagnose cancer by analyzing the expression patterns of various miRNAs.

Diagnosis using miRNAs may generally be performed by comparing differences in expression levels. However, since expression levels vary slightly from person to person and there is a lack of micro-RNAs that are always expressed consistently, diagnosis using absolute values may be less accurate. In addition, judging by comparing the relative expression levels as described in Example 5 has the disadvantage of being cumbersome and time-consuming. Therefore, the present inventors attempted to derive results simply and quickly while increasing accuracy through machine learning using the Ct value data obtained through Example 5.

Specifically, a trained artificial intelligence model was constructed by training an artificial neural network (ANN) model using data from a total of 200 people, including 150 cancer patients (50 lung cancer patients, 50 colorectal cancer patients, and 50 pancreatic cancer patients) and 50 normal people.

First, training was performed using 100 datasets randomly extracted from 200 datasets (one data set has Ct values for the 36 miRNAs) as training datasets, 50 datasets as validation datasets, and 50 datasets as test datasets. Output class label were assigned as follows: 1: lung cancer, 2: colorectal cancer, 3: pancreatic cancer, and 4: normal. The artificial neural network model was composed of a single-layer feedforward network with one hidden layer, ReLU was used as the activation function, and there were 40 hidden nodes (neurons) in the hidden layer.

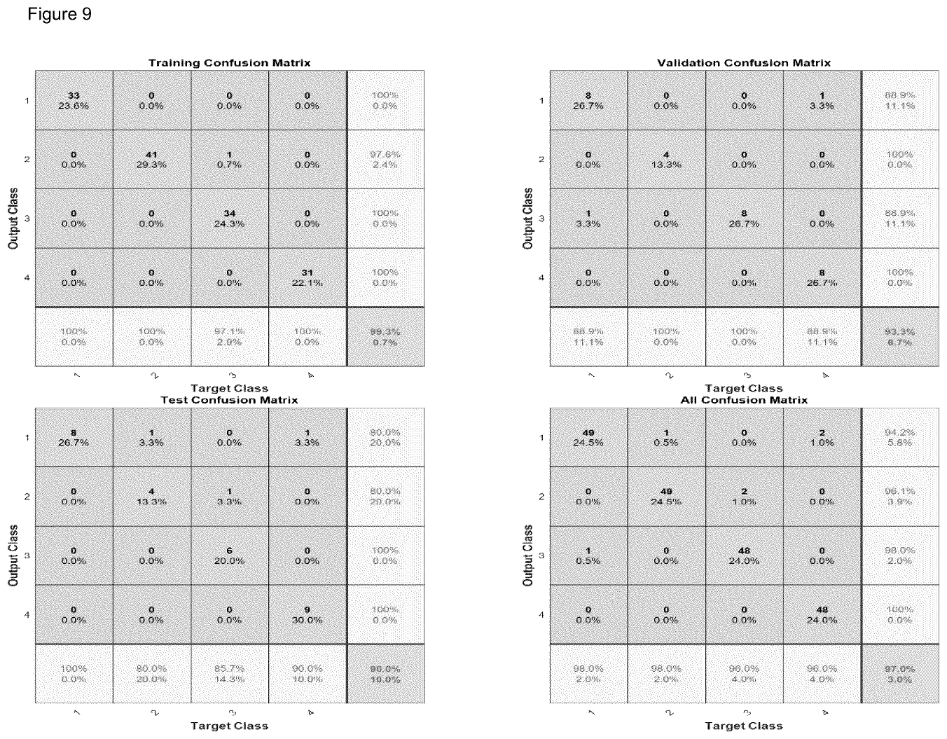

Next, the second training was performed using 140 datasets randomly extracted from 200 datasets as training datasets, 30 datasets as validation datasets, and 30 datasets as test datasets. As a result, the final accuracy was found to be 97% (see FIG. 9).

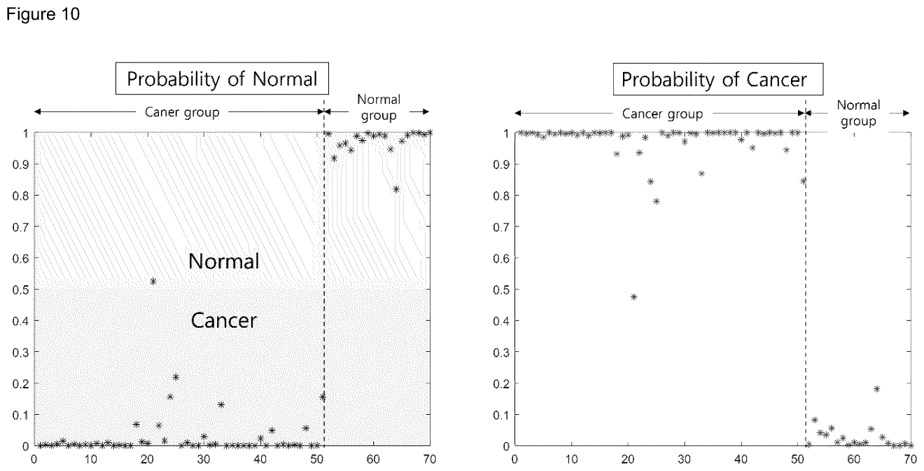

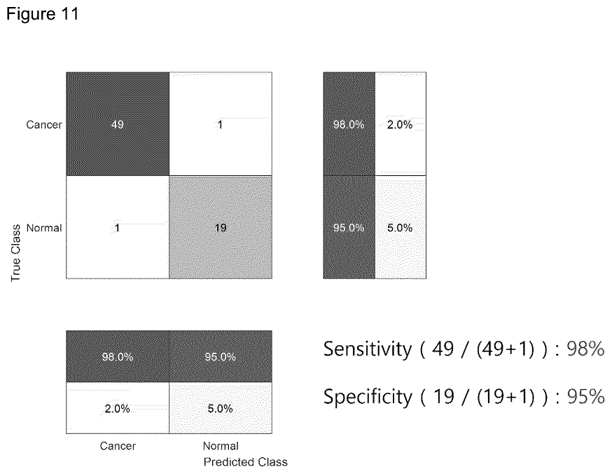

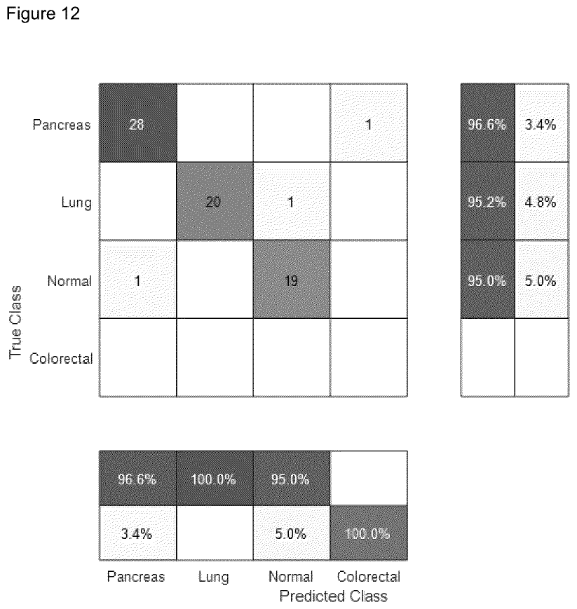

As a result of inputting into the trained artificial intelligence model the Ct values for 36 miRNAs derived by the biochip system for 50 cancer patients and 20 normal people as one dataset and obtaining an output from trained artificial intelligence model, 49 out of 50 cancer patients and 19 out of 20 normal people were correctly diagnosed as cancer patients and normal people, showing 98% sensitivity and 95% specificity (see FIGS. 10 and 11). In addition, in distinguishing cancer types, 1 out of 20 normal people was misdiagnosed with pancreatic cancer, and 19 people were judged as normal; 1 out of 21 lung cancer patients was misdiagnosed as normal, and 20 people were correctly diagnosed with lung cancer; and 1 out of 29 patients with pancreatic cancer was misdiagnosed with colorectal cancer, and 28 patients were correctly diagnosed with pancreatic cancer, showing a high accuracy of 95.7% in the ability to distinguish between cancer types (see FIG. 12).

Therefore, using the constructed artificial intelligence model, it will be possible to accurately diagnose and predict prognosis of a cancer type from the Ct values of 36 miRNA for a sample.

What is claimed is:

1. A method of providing information for diagnosis or prognostic prediction of multiple types of cancer comprising steps of:
detecting multiple biomarkers in a biological sample isolated from a subject;
comparing and classifying detection information of the multiple biomarkers detected in the above step of detecting multiple biomarkers; and
determining the type of cancer of the sample based on the detection information of multiple biomarkers classified in the above step of comparing and classifying detection information,
wherein the step of detecting multiple biomarkers is a step of detecting the presence or absence of the biomarkers in the sample, or measuring levels of the biomarkers, or performing both,
the comparing and classifying comprises comparing the detection information of multiple biomarkers detected from the sample with detection information of multiple biomarkers in normal people shown in Table 1 below and classifying the detection information of multiple biomarkers detected from the sample into four groups: increase by 2-fold or more, increase by less than 2-fold, decrease by less than ½-fold, and decrease by more than ½-fold, compared to the detection information of multiple biomarkers in normal people,
the detection information of multiple biomarkers in normal people is information shown in Table 1 below, and
the determining the type of cancer comprises: (1) determining that a case, in which at least 6 of biomarkers specific to each cancer type have the same detection information as criteria shown in Table 13 below while at least 20 of the multiple biomarkers have the same detection information as criteria shown in Table 13 below, is a corresponding cancer type; or determining that (2) a case, in which increases in detection information (e.g., expression levels) of at least 5 biomarkers selected from the group consisting of miR-139-3p, miR-29c-3p, miR-148a-3p, miR-30a-5p, miR-210-5p, miR-150-3p, miR-181b-5p, miR-34a-3p, miR-29a-3p, miR-145-5p, miR-26b-3p, miR-221-3p, and miR-431 are 2-fold or more while at least 20 of the multiple biomarkers have the same detection information as the criteria shown in Table 13 below, is pancreatic cancer among multiple types of cancers:
TABLE 1

| miRNA | Ct value | miRNA | Ct value | miRNA | Ct value |
|---|---|---|---|---|---|
| miR-34b-5p | 35.8 | miR-26a-5p | 24.8 | miR-6761 | 33.1 |
| miR-124a-3p | 32.5 | miR-148a-3p | 26.9 | miR-339-3p | 30.9 |
| miR-1-3p | 29.7 | miR-30a-5p | 25.7 | miR-126-3p | 25.8 |
| miR-146b-5p | 30.2 | miR-210-5p | 32.3 | miR-34a-3p | 30.0 |
| miR-26b-5p | 24.4 | miR-150-3p | 32.6 | miR-29a-3p | 25.2 |
| miR-92a-3p | 23.6 | miR-155-3p | 35.6 | miR-145-5p | 28.3 |
| miR-142-3p | 25.1 | miR-16b-5p | 21.2 | miR-18a-5p | 26.9 |
| miR-139-3p | 31.1 | miR-181b-5p | 32.3 | miR-26b-3p | 32.3 |
| miR-25-3p | 26.0 | miR-223-3p | 24.3 | miR-21-5p | 24.2 |
| miR-15b-5p | 25.1 | miR-483-3p | 28.7 | miR-221-3p | 25.4 |
| miR-29b-3p | 25.1 | miR-505-5p | 33.6 | miR-431 | 32.3 |
| miR-29c-3p | 24.2 | miR-636-5p | 33.3 | miR-103-3p | 24.2 |

TABLE 13

| | Lung cancer | Colorectal cancer | Pancreatic cancer |
|---|---|---|---|
| miR-34b-5p | + | + | ++++ |
| miR-124a-3p | + | + | + |
| miR-1-3p | ++ | + | ++++ |
| miR-146b-5p | ++++ | ++ | ++++ |
| miR-26b-5p | ++++ | ++++ | ++++ |
| miR-92a-3p | ++++ | ++++ | ++ |
| miR-142-3p | ++++ | ++++ | ++++ |
| miR-139-3p | + | + | ++++ |
| miR-25-3p | ++++ | ++++ | ++++ |
| miR-15b-5p | ++++ | ++++ | ++++ |
| miR-29b-3p | ++++ | ++++ | ++++ |
| miR-29c-3p | | ++ | ++++ |
| miR-26a-5p | ++++ | +++ | ++++ |
| miR-148a-3p | ++ | + | ++++ |
| miR-30a-5p | ++ | + | ++++ |
| miR-210-5p | + | + | ++++ |
| miR-150-3p | + | + | ++++ |
| miR-155-3p | + | ++ | + |
| miR-16b-5p | ++++ | ++++ | +++ |
| miR-181b-5p | | + | ++++ |
| miR-223-3p | +++ | +++ | ++++ |
| miR-483-3p | + | + | + |
| miR-505-5p | ++++ | ++++ | ++++ |
| miR-636-5p | ++ | +++ | ++ |
| miR-6761 | + | + | +++ |
| miR-339-3p | +++ | ++ | ++++ |
| miR-126-3p | ++++ | | ++++ |
| miR-34a-3p | + | + | ++++ |
| miR-29a-3p | + | + | ++++ |
| miR-145-5p | + | + | ++++ |
| miR-18a-5p | ++++ | ++++ | ++++ |
| miR-26b-3p | ++ | + | ++++ |
| miR-21-5p | +++ | + | ++++ |
| miR-221-3p | + | + | ++++ |
| miR-431 | | + | ++++ |
| miR-103-3p | ++++ | ++++ | ++++ |
| GAPDH | ++++ | + | ++++ |

in Table 13 above, ++++ denotes a case in which the relative increase in the expression level of the biomarker relative to the expression level of the biomarker in normal people is 2-fold or more, +++ denotes a case in which the relative increase in the expression level of the biomarker relative to the expression level of the biomarker in normal people is less than 2-fold, ++ denotes a case in which the relative decrease in the expression level of the biomarker relative to the expression level of the biomarker in normal people is less than ½-fold, and + denotes a case in which the relative decrease in the expression level of the biomarker relative to the expression level of the biomarker in normal people is ½-fold or more.

2. The method according to claim 1, wherein the multiple biomarkers are miR-1-3p, miR-139-3p, miR-124a-3p, miR-142-3p, miR-146b-5p, miR-15b-5p, miR-26b-5p, miR-25-3p, miR-34b-5p, miR-92a-3p, miR-126-3p, miR-148a-3p, miR-21-5P, miR-210-5p, miR-221-3p, miR-26a-5p, miR-29a-3p, miR-29b-3p, miR-29c-3p, miR-30a-5p, miR-34a-3p, miR-145-5p, miR-150-3P, miR-155-3P, miR-16b-5p, miR-18a-5p, miR-181b-5p, miR-223-3p, miR-26b-3p, miR-483-3p, miR-505-5p, miR-636-5p, miR-6761, miR-339-3P, miR-431, and miR-103-3p.

3. The method according to claim 1, wherein the multiple biomarkers specific to lung cancer are miR-1-3p, miR-139-3p, miR-124a-3p, miR-142-3p, miR-146b-5p, miR-126-3p, miR-148a-3p, miR-21-5P, miR-210-5p, miR-221-3p, miR-26a-5p, miR-181b-5p, miR-223-3p, miR-26b-3p, miR-6761, and miR-339-3P.

4. The method according to claim 1, wherein the multiple biomarkers specific to colorectal cancer are miR-1-3p, miR-15b-5p, miR-26b-5p, miR-25-3p, miR-34b-5p, miR-92a-3p, miR-18a-5p, miR-26b-3p, miR-483-3p, miR-505-5p, miR-636-5p, miR-6761, miR-339-3P, and miR-431.

5. The method according to claim 1, wherein the multiple biomarkers specific to pancreatic cancer are miR-29a-3p, miR-29b-3p, miR-29c-3p, miR-30a-5p, miR-34a-3p, miR-145-5p, miR-150-3p, miR-155-3P, miR-16b-5p, miR-181b-5p, miR-223-3p, miR-483-3p, miR-505-5p, miR-636-5p, miR-6761, and miR-339-3p.

6. The method according to claim 1, wherein the detecting the multiple biomarkers is performed by promers.

7. A method of providing information for diagnosis or prognostic prediction of multiple types of cancer comprising steps of:
detecting multiple biomarkers in a biological sample isolated from a subject; and
inputting detection information of the multiple biomarkers detected in step above into a trained artificial intelligence model and analyzing the input information,
wherein the above step of detecting multiple biomarkers is a step of detecting the presence or absence of the biomarkers in the sample, or measuring levels of the biomarkers, or performing both,
the trained artificial intelligence model is a model into which the detection information of biomarkers measured in the sample is input and which outputs, as an output value, determination of the type of cancer of the sample, obtained by and is a model classifying detection information of previously detected multiple biomarkers in normal people and patients into a training dataset, a validation dataset, and a test dataset, modeling a deep neural network (DNN) algorithm, and testing the modeled DNN algorithm, and
the modeled DNN algorithm learns and tries various parameters to find optimized parameters by applying hyper-parameters, and uses ReLU (Rectified Linear Unit) or TanH (Hyperbolic Tangent) an as activation function to compensate for gradient loss, learning convergence speed, and overfitting.

8. The method according to claim 7, wherein the detection information of the multiple biomarkers is expression levels of miR-1-3p, miR-139-3p, miR-124a-3p, miR-142-3p, miR-146b-5p, miR-15b-5p, miR-26b-5p, miR-25-3p, miR-34b-5p, miR-92a-3p, miR-126-3P, miR-148a-3p, miR-21-5P, miR-210-5p, miR-221-3p, miR-26a-5p, miR-29a-3p, miR-29b-3p, miR-29c-3p, miR-30a-5p, miR-34a-3p, miR-145-5p, miR-150-3P, miR-155-3P, miR-16b-5p, miR-18a-5p, miR-181b-5p, miR-223-3p, miR-26b-3p, miR-483-3p, miR-505-5p, miR-636-5p, miR-6761, miR-339-3P, miR-431, and miR-103-3p.

9. The method according to claim 7, wherein the detecting the multiple biomarkers is performed by promers.
